(19) United States
(12) Patent Application Publication
(10) Pub. No.: US 2005/0096323 A1
(43) Pub. Date: May 5, 2005

(54) Diketopiperazine derivatives to inhibit thrombin

(75) Inventors: Yudu Cheng, Cote Saint-Luc (CA); Jeffrey Manwell, Ottawa (CA)

Correspondence Address: OGILVY RENAULT, 1981 MCGILL COLLEGE AVENUE, SUITE 1600, MONTREAL, QC, H3A2Y3, CA

(73) Assignee: NovoScience Pharma Inc., Montreal (CA)

(21) Appl. No.: 10/937,574

(22) Filed: Sep. 10, 2004

Related U.S. Application Data
(63) Continuation-in-part of application No. 10/130,281, filed on Aug. 28, 2002, now abandoned.
(60) Provisional application No. 60/167,901, filed on Nov. 30, 1999.
(60) Provisional application No. 60/194,366, filed on Apr. 4, 2000.

(51) Int. Cl. A61K 31/498; C07D 487/04

(52) U.S. Cl. 514/249; 544/349

(57) ABSTRACT

The present invention relates to compounds to inhibit blood coagulation, and more particularly to novel diketopiperazine derivatives, pharmaceutically acceptable salts and compositions thereof, to specifically inhibit thrombin. The compound has the following general structure 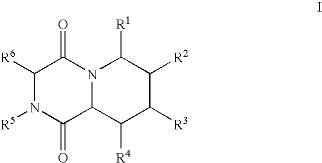
wherein R¹, R² and R⁴ consist of a hydrogen, alkyl or aryl moiety, R³ consist of an alkyl or aryl moiety, wherein R⁵ consists of a hydrogen, alkyl, aryl, hydroaryl, heteroaryl, hydroheteroaryl, sulfonylalkyl, sulfonylaryl, sulfonylhydroaryl, sulfonylheteroaryl or sulfonylhydroheteroaryl moiety, and wherein R⁶ consists of a hydrogen, alkyl, aryl, hydroaryl, heteroaryl or hydroheteroaryl moiety. Also disclosed are methods of using the compound for treating coagulation disorders such as thrombosis and heparin associated thrombocytopenia.

### CROSS-REFERENCE TO RELATED APPLICATIONS

This application is a continuation-in-part of U.S. application Ser. No. 10/130,281 filed Aug. 28, 2002 entitled “DIKETOPIPERAZINE DERIVATIVES TO INHIBIT THROMBIN” which is a National Stage under 35 U.S.C 371 of PCT/CA00/01414 filed Nov. 29, 2000 which claims the benefit of U.S. provisional applications Nos. 60/167,901 filed Nov. 30, 1999 and 60/194,366 filed Apr. 4, 2000.

### BACKGROUND OF THE INVENTION

(a) Field of the Invention

The present invention relates to compounds to inhibit blood coagulation, and more particularly to diketopiperazine derivatives, pharmaceutically acceptable salts and compositions thereof, to specifically inhibit thrombin.

(b) Description of Prior Art

Thrombotic disorders are characterized by the formation of a thrombus obstructing the vascular blood flow, causing arterial or venous thrombosis or thromboembolism. Thrombi are composed of fibrin, platelets, white blood cells (WBCs) and red blood cells (RBCs). Thrombus formation involves several genetic and environmental factors. Genetically impaired anticoagulant mechanisms include factor V resistance to activated protein C, hyperhomocysteinemia, protein C deficiency, protein S deficiency, antithrombin deficiency and defective fibrinolysis, while thrombotic stimuli include surgery, pregnancy, oral contraceptive use and antiphospholipid antibodies. Chronic and acute thrombotic complications, including venous and arterial thrombosis, atrial fibrillation, stroke, myocardial infarction and pulmonary embolism are the leading cause of deaths worldwide.

Antithrombotic therapy involves thrombolytic drug therapy, to remove thrombi, and the use of antiplatelet drugs and anticoagulants, to inhibit coagulation. Subsequent therapy varies depending on the venous or arterial circulatory system involved and the size and location of the vessels.

The anticoagulant drugs currently used show several disadvantages (Exp. Opin. Inves. Drugs 1997, 6:1591-1622; Current Pharmaceutical Design 1995, 1:441-468; Circulation 1994, 90:1522-1536).

For example, heparin is the first agent to be administered parenterally in situations requiring acute anticoagulation. Heparin consists of a mucopolysaccharide of animal origin. Standard or high molecular weight heparins (HMWHs) consist of molecules of many different sizes, while depolymerized or low molecular weight heparins (LMWHs) have a molecular weight between 4000 and 6000 D.

There are several disadvantages related to the use of heparin, namely (1) heparin is a parenteral agent requiring intravenous (i.v.) or sub-cutaneous (s.c.) administration; (2) the anticoagulant dose-response curve for heparin is not linear, and ex vivo coagulation parameters (APTT) must be followed to monitor the degree of anticoagulation; (3) heparin is ineffective at inhibiting clot-bound thrombin; and (4) there are reports of a “rebound” reactivation of unstable angina subsequent to discontinuation of heparin therapy, and heparin has been associated with thrombocytopenia, requiring monitoring of platelet counts. Heparin-induced thrombocytopenia (HIT) is an immunoglobulin-mediated adverse drug reaction that is characterized by platelet activation, thrombocytopenia, and a high risk of thrombotic complications among patients receiving or who have recently received heparin.

In the case of venous thrombosis or pulmonary embolism, a 7-10 day course of parenteral heparin is usually followed by prolonged administration of the only currently available oral anticoagulant drug, warfarin, to prolong treatment of thrombotic complications. Heparin is generally coadministered with warfarin for a few days prior to cessation of heparin therapy.

Warfarin has several disadvantages (The Annals of Pharmacotherapy 1995, 29:1274-1282; Clin. Pharmacokinet. 1996, 30:416-444), namely: (1) it carries a risk of bleeding, (2) it exhibits adverse drug and diet interactions, and (3) it requires frequent monitoring.

Both heparin and warfarin are indirect anticoagulants and their functions depend on the presence of antithrombin and vitamin K, respectively. Consequently, following the cessation of warfarin treatment, one has to wait for the resynthesis of vitamin K-dependent coagulation factors by the liver to restore the haemostatic balance. These drawbacks limit the physician acceptance and usage of warfarin in treating thrombotic disorders.

The liabilities of the conventional anticoagulant therapy have prompted the development of novel anticoagulants over the last two decades. LMWHs were discovered to have similar efficacy to the heparin in 1976. Their favorable pharmacokinetic profiles and risk/ratios have led to widespread use in Europe since 1992 and, more recently, approval for their use in USA.

The new anticoagulant strategy is based on direct inhibition of critical enzymes in the coagulation cascade. As the final enzyme in the coagulation cascade, thrombin has been an extensively tested target. Thrombin, the key regulator of the thrombotic process, is a trypsin-like serine protease. Thrombin has many and varied biological functions, but its main action is to catalyze the transformation of fibrinogen to fibrin, whether the thrombin is soluble in plasma or fibrin-bound. Fibrin is then polymerized and cross-linked by the action of activated blood factor XIII to form insoluble blood clotting. Thrombin also activate blood factors V and VIII which in turn accelerates the blood coagulation by a feed-back mechanism. As a potent activator of platelets, thrombin also plays an important role in driving the growth of platelet-rich thrombi in the arterial circulation. The fibrin deposition and platelet aggregation can thus be interrupted when thrombin is inhibited. However, thrombin is similar to numerous serine-proteases present in the human body and particularly in the blood, such as plasmin. A thrombin inhibitor therefore needs to be specific to thrombin.

Thrombin inhibitors can directly inactivate thrombin by binding to thrombin active site and/or fibrinogen recognition exosite (FRE), whereby fibrinogen is transformed into fibrin. For example, hirudin is a naturally occurring 63-amino acid anticoagulant which is produced in the salivary glands of the blood sucking leech Hirudo medicinalis. Hirudin inhibits thrombin by directly binding both the thrombin active site and the FRE with an inhibition constant (Kᵢ) value of 2.0×10⁻¹⁵M against thrombin (Biochemistry 1986, 25:4622-4628). Hirugen is a peptide derived from the anionic carboxy-terminus of hirudin and binds only the FRE of thrombin with a Kᵢ value of 1.44×10⁻⁷M (J. Biol. Chem. 1989, 264:8692-8698). Hirulogs or Bivalirudin is a synthetic peptide consisting of a hirugen-like FRE-binding sequence linked by a glycine-spacer to the substrate-like active-site binding moiety, D-phenyalanine-prolyl-arginine with a Kᵢ value of 2.3×10⁻⁹M.

Except for a slightly better safety profile in terms of the bleeding complications, the above-mentioned direct thrombin inhibitors have shown no better and even worse features than heparin, namely (1) relatively short half lives, (2) parenteral administration, and (3) cost-ineffectiveness.

U.S. Pat. Nos. 4,258,192 and 4,201,863 disclose a synthetic small molecule thrombin inhibitor with a Kᵢ value of 1.9×10⁻¹⁰M for human thrombin, which is commercialized as Argatroban (Novastan, Mitsubishi Chemical Corp Cardiovasc. Drug Rev. 1991, 9:247-263). It was developed for the indications of chronic arterial obstruction, acute ischaemic stroke and haemodialysis in antithrombin III (ATIII)-deficient patients, and as a replacement for heparin in patients at risk of (HIT). However, Argatroban is still not an ideal small molecule thrombin inhibitor due to the following problems: it is (1) not orally bioavailable; (2) less effective on venous than arterial thrombosis; (3) possibly dose-dependent; (4) thrombin rebound effect; (5) not more effective than heparin in treatment of unstable angina, coronary angioplasty and acute myocardial infarction.

It would therefore be highly desirable to be provided with an orally available thrombin inhibitor. The interest in orally bioavailable thrombin inhibitors is high (Am. J. Cardiol. 1995, 75:27B-33B). A small molecule thrombin active site inhibitor that would selectively and reversibly inhibit thrombin would present a distinct advantage over warfarin with respect to side-effects and monitoring, as described above. The selectivity of a thrombin inhibitor compared to warfarin would allow it to be used with relative safety in both arterial and venous thrombosis. Another distinct advantage of a small molecule thrombin inhibitor would be its potentially important ability to inhibit clot-bound thrombin as well as fluid-phase thrombin.

### SUMMARY OF THE INVENTION

One aim of the present invention is to provide an orally available specific thrombin inhibitor.

In accordance with the present invention there is provided a compound of the following formula I: 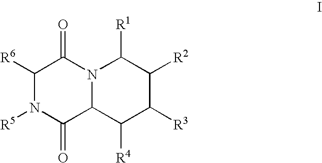
or a pharmaceutically acceptable salt or stereoisomer thereof, wherein R¹, R² and R⁴ consist of a hydrogen, alkyl or aryl moiety, R³ consist of an alkyl or aryl moiety, R⁵ consists of a hydrogen, alkyl, aryl, hydroaryl, heteroaryl, hydroheteroaryl, sulfonylalkyl, sulfonylaryl, sulfonylhydroaryl, sulfonylheteroaryl or sulfonylhydroheteroaryl moiety, and R⁶ consists of a hydrogen, alkyl, aryl, hydroaryl, heteroaryl or hydroheteroaryl moiety. Such a compound inhibits thrombin or blood coagulation and may be used as an antithrombotic or an anticoagulant.

For example, R⁵ may consist of an alkyl, aryl, hydroaryl, heteroaryl or hydroheteroaryl moiety. More particularly, R¹, R² and R⁴ may consist of a hydrogen moiety, R³ may consist of a methyl moiety, R⁵ may consist of 1,2,3,4-tetrahydro-3-methyl-8-quinolinesulfonyl, and R⁶ may consist of 3-guanidinopropyl. Such a compound has a high inhibition constant (Kᵢ is 5.3×10⁻¹⁵M).

In accordance with the present invention, there is further provided a pharmaceutical composition comprising such a compound as an active ingredient, in association with a pharmaceutically acceptable carrier. The pharmaceutical composition may be suitable for oral administration. The active ingredient may be used in a composition such as a tablet, capsule, solution or suspension containing about 5 to about 500 mg per unit of dosage of a compound of formula I or a mixture thereof. The compounds may be combined in a conventional manner with a physiologically acceptable vehicle or carrier including suitable expedients, binders, preservatives, stabilizers, flavors, etc. as accepted in the pharmaceutical practice.

In accordance with the present invention, there is further provided a method for substantially preventing thrombin activity in a mammal or a human or a tissue thereof. The method comprises administering an effective amount of such the compound or the pharmaceutical composition to the mammal, human or tissue.

In accordance with the present invention, there is further provided a method for treating a coagulation disorder in a mammal or a human or a tissue thereof. The method comprises administering an effective amount of the compound or the pharmaceutical composition to the mammal, human or tissue. Examples of coagulation disorders include thrombosis or heparin-induced thrombocytopenia (HIT).

In an aspect of the present invention, the need of an individual for treatment and the efficacy of the treatment can be assessed, for example, by measuring the activated partial thromboplastin time (APTT), clot retraction time (CT), thrombin time (TT) prothrombin time (PT) or any other test that would be indicative of coagulation disorders and as would be known to one skilled in the art.

The dosage ranges for the administration of the compounds used in the present invention are those large enough to achieve the desired effect. The dosage may vary for example, with condition and age of the subject and the extent of the coagulation disorder and can be determined by those skilled in the art.

In one embodiment of the invention, the compounds of the present invention may be administered orally to various mammalians known to thrombotic disorders, such as humans, cats, dogs, monkeys, mice and the like in an effective dosage range of 0.1 to 100 mg/kg, preferably about 0.2 to 50 mg/kg and more preferably about 0.5 to 25 mg/kg on a regimen in single or 2 to 4 divided daily doses.

Although the compound of formula I of the present invention inhibits thrombin and may be used as an anticoagulant, it may also be used in combination with other antithrombotic or anticoagulant drugs.

The compound of the present invention comprises an extra ring and substitutions to a diketopiperadine structure, and is more rigid compared to known diketopiperadine derivatives.

The compound of the present invention inhibits blood coagulation by specifically binding to thrombin. Compared to anticoagulant drugs such as Heparin, Warfarin, Hirudin, Hirugen, Hirulogs and Argatroban. The compound of the present invention exhibits oral bioavailability, an increased half-life, effectiveness on venous thrombosis and limited or no “rebound” effect on thrombin, contrary to heparin. The compound of the present invention also reduces the risk of heparin-induced thrombocytopenia (HIT).

For the purpose of the present invention, the following terms are defined below.

The term “alkyl” is intended to mean a straight or a branched chain radical(s) or cyclic ring(s) of up to 18 carbons, preferably of 1 to 8 carbons, such as methyl, ethyl, propyl, butyl, pentyl, hexyl, heptyl, octyl, nbnyl, decyl, undecyl, dodecyl, and the various branched chain isomers thereof and/or 1 or 2 of the following substituents: an aryl substituent (for example, to form benzyl or phenethyl), a cycloalkyl substituent, an alkylcycloalkyl substituent, an alkenyl substituent, an alkynyl substituent, hydroxy, alkoxy, halogen, amino, alkylamino, dialkylamino, guanidino or carboxy substituent.

The term “aryl” is intended to mean a monocyclic, bicyclic or tricyclic aromatic group(s) containing 6 to 14 carbons in the ring portion, such as phenyl, naphtyl or anthracenyl. The aryl moiety may include substituted aryl, which may include 1 or 2 substituents such as alkyl, cyano, amino, alkylamino, dialkylamino, nitro, carboxy, carboalkoxy, trifluoromethyl, halogen, alkoxy, arylalkoxy or hydroxy.

The term “hydroaryl” is intended to mean 10 to 14-membered aromatic rings, such as tetrahydronapthyl, tetrahydroanthracenyl and the like. Hydroaryl may include substituted hydroaryl, which may include 1 or 2 substituents such as alkyl, cyano, amino, alkylamino, dialkylamino, nitro, carboxy, carboalkoxy, trifluoromethyl, halogen, alkoxy, arylalkoxy or hyroxy.

The term “heteroaryl” is intended to mean 5- to 14-membered aromatic ring(s) which includes 1,2 or 3 heteroatoms such as nitrogen, oxygen or sulfur, such as 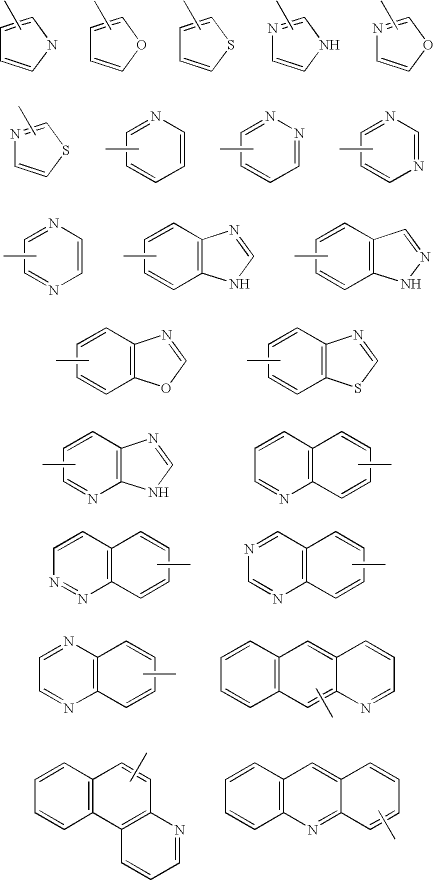
and the like. The heteroaryl rings may optionally be fused to aryl rings such as defined previously. The heteroaryl rings may include a substituted heteroaryl, which may include 1 or 2 substituents such as alkyl, cyano, amino, alkylamino, dialkylamino, nitro, carboxy, carboalkoxy, trifluoromethyl, halogen, alkoxy, arylalkoxy or hyroxy.

The term “hydroheteroaryl” is intended to mean a reduced form of the above-mentioned heteroaryl rings, such as: 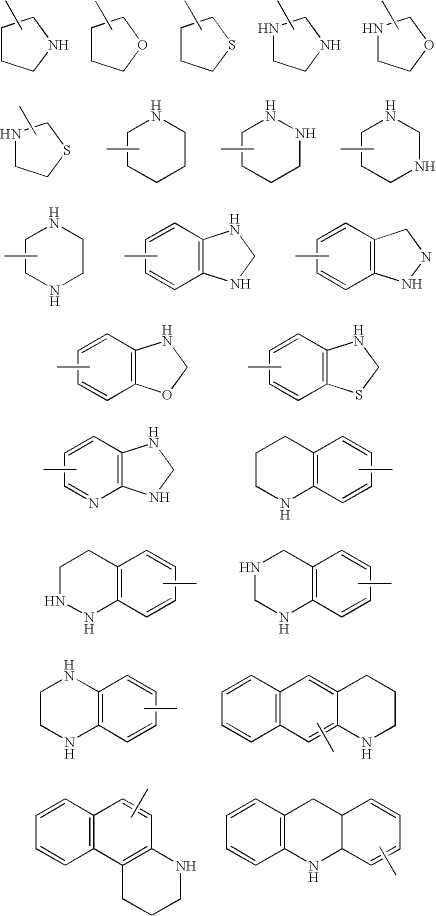
and the like. The hydroheteroaryl rings may optionally be fused to aryl rings such as defined previously. The hydroheteroaryl rings may include substituted hydroheteroaryl, which may include 1 or 2 substituents such as alkyl, cyano, amino, alkylamino, dialkylamino, nitro, carboxy, carboalkoxy, trifluoromethyl, halogen, alkoxy, arylalkoxy or hyroxy.

The term “sulfonylalkyl” is intended to mean a sulfonyl group (SO₂) in which an alkyl group such as defined previously is attached.

The term “sulfonylaryl” is intended to mean a sulfonyl group (SO₂) in which an aryl group such as defined previously is attached.

The term “sulfonylhydroaryl” is intended to mean a sulfonyl group (SO₂) in which an hydroaryl group such as defined previously is attached.

The term “sulfonylheteroaryl” is intended to mean a sulfonyl group (SO₂) in which a heteroaryl group such as defined previously is attached.

The term “sulfonylhydroheteroaryl” is intended to mean a sulfonyl group (SO₂) in which a hydroheteroaryl group such as defined previously is attached.

A pharmaceutically acceptable acid salt may be obtained from the compound of formula I of the present invention by reacting the free base with an acid, such as hydrochloric, hydrobromic, hydroiodic, nitric, sulfuric, phosphoric, acetic, citric, maleic, succinic, lactic, tartaric, gluconic, benzoic, methanesulfonic, ethanesulfonic, benzenesulfonic, p-toluenesulfonic acid or the like.

### BRIEF DESCRIPTION OF THE DRAWINGS

FIG. 1 illustrates a progressive curve of an in vivo thrombin inhibition assay at the substrate concentration ([S]) of 2.5 μM by the compound of cycloargatroban (formula I) where R¹, R² and R⁴ are hydrogen, R³ is Me=CH₃, R⁵ is 1,2,3,4-tetrahydro-3-methyl-8-quinolinesulfonyl and R⁶ is 3-guanidinopropyl.

FIG. 2 illustrates a progressive curve of an in vivo thrombin inhibition assay at the substrate concentration ([S]) of 10 μM by the compound of cycloargatroban (formula I) where R¹, R² and R⁴ are hydrogen, R³ is Me=CH₃, R⁵ is 1,2,3,4-tetrahydro-3-methyl-8-quinolinesulfonyl and R⁶ is 3-guanidinopropyl.

FIG. 3 illustrates a progressive curve of an in vivo trypsin inhibition assay at the substrate concentration ([S]) of 22 μM by the compound of cycloargatroban (formula I) where R¹, R² and R⁴ are hydrogen, R³ is Me=CH₃, R⁵ is 1,2,3,4-tetrahydro-3-methyl-8-quinolinesulfonyl and R⁶ is 3-guanidinopropyl.

FIG. 4 illustrates an in vivo kinetic assay to determine the inhibition constant (Ki) for thrombin by the compound of cycloargatroban (formula I) where R¹, R² and R⁴ are hydrogen, R³ is Me=CH₃, R⁵ is 1,2,3,4-tetrahydro-3-methyl-8-quinolinesulfonyl and R⁶ is 3-guanidinopropyl.

FIG. 5 illustrates an ex vivo coagulation assay of the compound of cycloargatroban (formula I) where R¹, R² and R⁴ are hydrogen, R³ is Me=CH₃, R⁵ is 1,2,3,4-tetrahydro-3-methyl-8-quinolinesulfonyl and R⁶ is 3-guanidinopropyl and the reference compound of argatroban with the chemical composition shown in structure XIII below.

### DETAILED DESCRIPTION OF THE INVENTION

In accordance with the present invention, there are provided compounds which are useful as potent and specific inhibitors of thrombin and blood coagulation in vitro and in vivo in mammals.

The invention involves the preparation of diketopiperazine derivatives of the formula I:

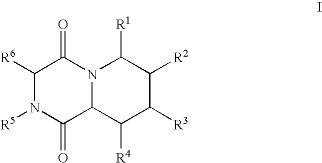

The compounds of formula I of the invention may be prepared according to the following Reaction Sequence I:

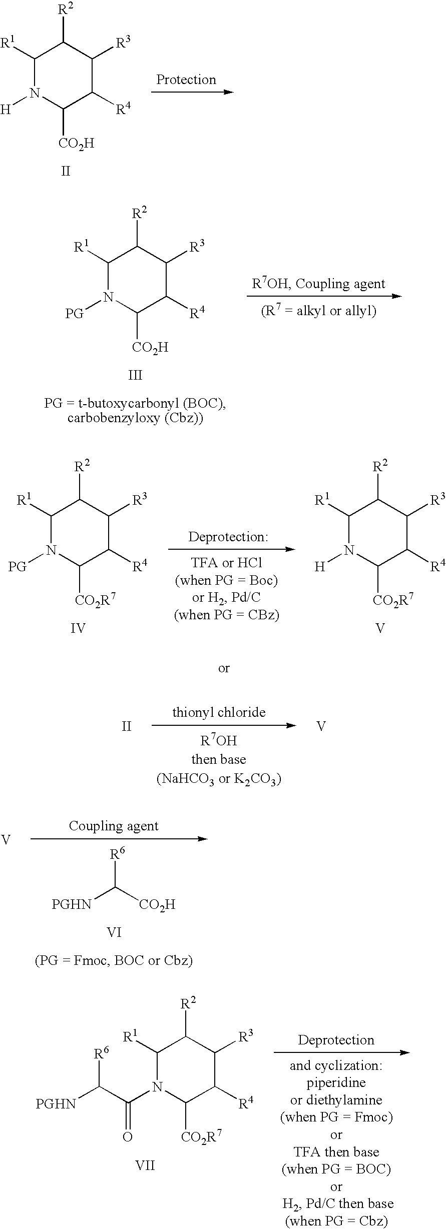

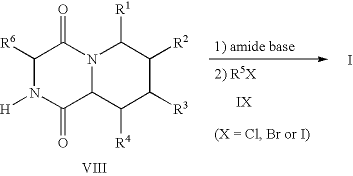

The amino acid II is protected with a tert-butoxycarbonyl group (BOC) using di-tert-butyl dicarbonate in 10% triethylamine (TEA) in methanol, or with a benzyloxycarbonyl group (Cbz) using benzyl chloroformate and aqueous sodium hydroxide solution in an organic solvent such as dioxane, tetrahydrofuran (THF) or ether. The protected amino acid III is esterified using a coupling reaction with an alcohol in the presence of dicyclohexylcarbodiimide (DCC), diisopropylcarbodiimide (DIC) or 2-(1H-benzotriazole-1-yl)-1,1,3,3-tetramethyluronium tetrafluoroborate (TBTU) and 4-dimethylaminopyridine (DMAP) or N-hydroxybenzotriazole (HOBT) and in the presence of an inert organic solvent such as dimethylformamide (DMF), N-methyl pyrrolidinone (NMP), dichloromethane (DCM) or THF at temperatures within the range of −20° C. to −5° C., to form an ester IV.

The ester IV is deprotected by treatment with trifluoroacetic acid (TFA) or hydrochloric acid (HCl) in the presence of a dry inert solvent such as DCM, THF, ethyl acetate or chloroform (BOC), or by hydrogenation over palladium on carbon in an alcoholic solvent (Cbz) at ambient temperature. Alternatively, the ester V may be prepared by the addition of thionyl chloride to an alcoholic solution of amino acid II at a temperature range within 0° C. to 20° C. followed by neutralization with a base such as sodium bicarbonate or potassium carbonate and the like. The ester V is made to undergo a coupling reaction with a protected amino acid derivative VI in the presence of a coupling reagent such as DCC, DIC or TBTU, and DMAP or HOBT, and a tertiary organic amine base such as TEA or diisopropylethylamine (DIPEA), and in the presence of an inert organic solvent such as DMF, NMP, THF or DCM at temperatures within the range of 0° C. to 20° C. to form the peptide VII. The peptide VII is deprotected and cyclized in the presence of piperidine or diethylamine, and an inert organic solvent such as DMF, NMP, DCM or THF and at ambient temperature (Fmoc), or deprotected by treatment with TFA or HCl in the presence of a dry inert solvent such as DCM, THF, ethyl acetate or chloroform (BOC), or by hydrogenation over palladium on carbon in an alcoholic solvent (Cbz) at ambient temperature, followed by addition of base to cause cyclization. The diketopiperazine VIII is treated with an amide organic base such as lithium bis (trimethylsilyl)amide (LHMDS) or lithium diisopropylamide (LDA), and in dry THF solvent at 0° C., followed by the addition of an alkylating agent IX at a temperature within the range of 0° C. and 20° C. to form the diketopiperazine I.

The compounds of formula I of the present invention may also be prepared according to the following Reaction Sequence II:

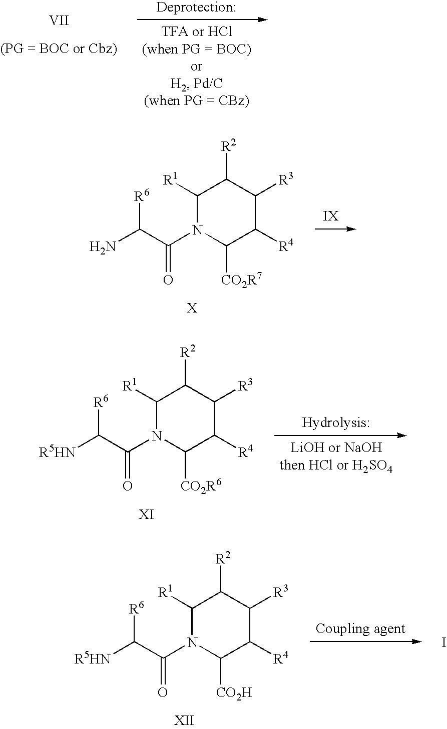

The peptide VII wherein PG is BOC or Cbz, is deprotected by treatment with TFA or HCl in the presence of a dry inert solvent such as DCM, THF, ethyl acetate or chloroform at ambient temperature (BOC), or by hydrogenation over palladium on carbon in an alcoholic solvent (Cbz). The peptide X is made to undergo a reaction with an alkylating agent IX, in the presence of a tertiary organic amine base such as pyridine, TEA or DIPEA, and in the presence of a dry inert solvent such as DCM, THF or chloroform at ambient temperature to form a peptide XI. The ester of peptide XI is hydrolyzed by treatment with an alkali metal base such as sodium hydroxide (NaOH) or lithium hydroxide (LiOH) in the presence of an alcohol solvent such as methanol or ethanol. The reaction mixture is acidified with HCl or sulfuric acid (H₂SO₄) to form an acid XII.

The acid XII is made to undergo an intramolecular cyclization reaction in the presence of TBTU, and HOBT, and DIPEA in an inert organic solvent such as DMF, NMP, THF or DCM at ambient temperature to form the diketopiperazine I.

The compounds of formula I of the present invention, wherein R⁶ is 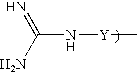
and Y is an alkyl, aryl, hydroaryl, heteroaryl or hydroheteroaryl moiety, may be prepared according to the following Reaction Sequence III:

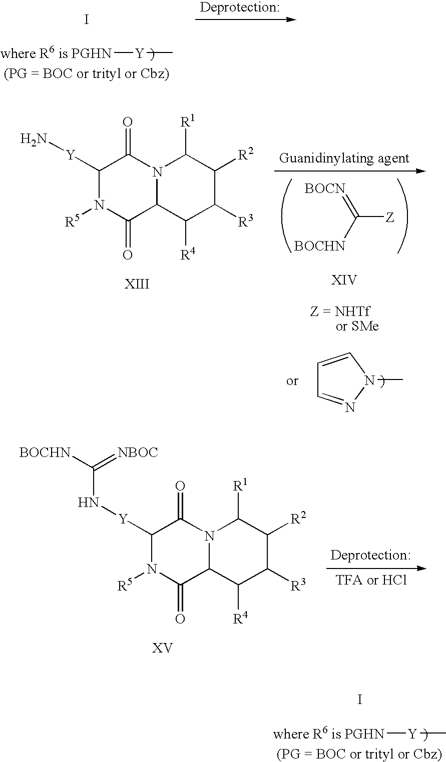

The diketopiperazine I is prepared following Reaction Sequence I or II wherein R⁶ is 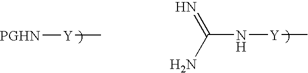
and deprotected by treatment with TFA or HCl in the presence of a dry inert solvent such as DCM, THF, ethyl acetate or chloroform (BOC, Trityl and the like), or by hydrogenation over palladium on carbon in an alcoholic solvent (Cbz) at ambient temperature. The diketopiperazine XIII is guanidinylated in the presence of guanidinylating reagents XIV such as N,N′-bis(tert-butoxycarbonyl)-N″-trifluromethanesulfonylguanidine, 1-[N,N′-bis(tert-butoxycarbonyl)amido]pyrazole or N,N′-bis(tert-butoxycarbonyl)-S-methylisothiourea, and a tertiary organic amine base such as TEA or DIPEA, and in the presence of an inert organic solvent such as DMF, NMP, THF or DCM at ambient temperature to form a protected guanidinylated diketopiperazine XV.

The diketopiperazine XV is deprotected by treatment with TFA or HCl in the presence of a dry inert solvent such as DCM, THF, ethyl acetate or chloroform at ambient temperature to form diketopiperazine I, wherein R⁶ is

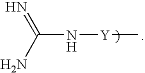

The compounds of formula I of the invention wherein R⁵ is hydroheteroaryl may be prepared according to the following Reaction Sequence IV.

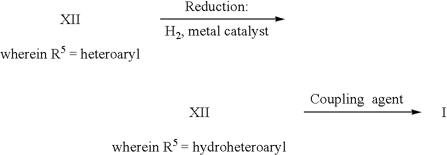

The acid XII is prepared following Reaction Sequence II wherein R⁵ is an aryl moiety and is subjected to a reduction in the presence of a catalyst containing metals such as palladium, platinum, rhodium or nickel, and at temperatures within the range of 20° C. to 100° C., and pressures within the range of 1 to 100 atmospheres to form the acid XII, wherein R⁵ is a hydroheteroaryl moiety. The acid XII wherein R⁵ is hydroheteroaryl is made to undergo an intramolecular cyclization reaction in the presence of a coupling agent TBTU, and HOBT, and DIPEA, and in the presence of an inert organic solvent such as DMF, NMP, THF or DCM at ambient temperature to form the diketopiperazine I, wherein R⁵ is hydroheteroaryl.

The present invention will be more readily understood by referring to the following examples, which are given to illustrate the invention rather than to limit its scope.

### EXAMPLE I

### N-(tert-Butoxycarbonyl)-D-2-piperidinecarboxylic acid, allyl ester

N-(tert-Butoxycarbonyl)-D-2-piperidinecarboxylic acid (2.0 g, 8.7 mmol, BACHEM) was dissolved in dichloromethane (40 mL), cooled to −20° C., allyl alcohol (1.0 ml, 15.0 mmol, Aldrich), dicyclohexylcarbodiimide (1.8 g, 8.7 mmol, Aldrich) and 4-dimethylaminopyridine (0.11 g, 0.87 mmol, Aldrich) were added and the reaction mixture was stirred between −5° C. and −10° C. for 4 h. After filtration to remove the urea byproduct, the reaction mixture was concentrated in vacuo. The resulting oil was subjected to chromatography on 100 g of silica gel and eluted with 15:1 hexane/ethyl acetate to give the title compound as a clear colorless liquid (2.33 g, 99%).

### EXAMPLE 2

### (2R,4R)-N-(tert-butoxycarbonyl)-4-methyl-2-piperidinecarboxylic acid, allyl ester

2R,4R)-4-Methyl-2-piperidinecarboxylic acid (250 mg, 1.75 mmol) was dissolved in 10% triethylamine in methanol (30 mL), cooled to 0° C. and di-tert-butyl dicarbonate (0.48 mL, 2.10 mmol, Aldrich) was added. After 2 h, the reaction mixture was concentrated in vacuo and sodium phosphate monobasic (10 mg) was added. The residue was dissolved in 1:1 ethyl acetate/water (10 mL) and the solution was adjusted to pH 2 with 1N hydrochloric acid. The mixture was extracted with ethyl acetate (4×20 mL) and the combined organic extracts were dried over anhydrous sodium sulfate and concentrated in vacuo. The resulting white solid was dissolved in dichloromethane (8 mL) and cooled to −20° C. Allyl alcohol (0.20 ml, 2.98 mmol, Aldrich), dicyclohexylcarbodiimide (361 mg, 1.75 mmol, Aldrich) and 4-dimethylaminopyridine (22 mg, 0.18 mmol, Aldrich) were added and the reaction mixture was stirred between −5° C. and −10° C. for 5 h. After filtration to remove the urea byproduct, the reaction mixture was concentrated in vacuo. The resulting oil was subjected to chromatography on 10 g of silica gel and eluted with 9:1 hexane/ethyl acetate to give the title compound as a clear colorless liquid (457 mg, 92%).

### EXAMPLE 3

### (3S,6R)-Bicyclo[4.4.0]-1,4-diaza-3-[(4-nitrophenyl)methyl]-4-N-(4-tert-butylbenzenesulfonyl)-2,5-decanedione, trifluroacetate salt

### 3A) 1-[N^{α}-(9-fluorenylmethoxycarbonyloxy)-L-4-nitrophenylalanyl]-D-2-piperidinecarboxylic acid, allyl ester

The pipecolic ester of Example 1 (259 mg, 0.96 mmol) was dissolved in 1:1 trifluroacetic acid/dichloromethane (5 mL) and stirred for 3 h. The reaction mixture was concentrated in vacuo and placed on a vacuum pump overnight. The resulting oil was dissolved in dimethylformamide (5 mL), cooled to 0° C. and diisopropylethylamine (0.50 mL, 2.88 mmol, Aldrich) was added. After stirring for 5 min, N^{α}-(9-fluorenylmethoxycarbonyloxy)-L-4-nitrophenylalanine (500 mg, 1.16 mmol, Novabiochem), N-hydroxybenzotriazole (205 mg, 1.34 mmol, Novabiochem) and 2-(1H-benzotriazole-1-yl)-1,1,3,3-tetramethyluronium tetrafluoroborate (430 mg, 1.34 mmol, Novabiochem) were added. The reaction mixture was stirred for 72 h, poured into ethyl acetate (125 mL) and washed with 10% hydrochloric acid (2×25 mL), saturated sodium bicarbonate solution (2×25 mL) and brine (25 mL). The organic layer was dried over anhydrous sodium sulfate and concentrated in vacuo. The resulting oil was subjected to chromatography on 50 g of silica gel and eluted with 7:3 hexane/ethyl acetate to give the title compound as a white solid (457 mg, 82%).

### 3B) (3S,6R)-Bicyclo[4.4.0]-1,4-diaza-3-[(4-nitrophenyl)methyl]-2,5-decanedione

To a solution of Part 3A ester (200 mg, 0.34 mmol) in dichloromethane (68 mL) was added piperidine (1.68 mL, 17.0 mmol, Aldrich) and the reaction mixture was stirred for 1 h. The reaction mixture was concentrated in vacuo and the resulting oil was subjected to chromatography on 20 g of silica gel and eluted with 19:1 dichloromethane/methanol to give the title compound as a pale yellow solid (69 mg, 67%).

### 3C) (3S,6R)-Bicyclo[4.4.0]-1,4-diaza-3-[(4-nitrophenyl)methyl]-4-N-(4-tert-butylbenzenesulfonyl)-2,5-decanedione, trifluroacetate salt

To a solution of Part 3B diketopiperazine (20 mg, 0.066 mmol) in anhydrous tetrahydrofuran (1 mL, Aldrich) under a nitrogen atmosphere at 0° C. was added 1.0 M lithium bis(trimethylsilyl)amide in tetrahydrofuran (0.090 mL, 0.090 mmol, Aldrich) and the reaction mixture was stirred for 1 h. 4-tert-Butylbenzenesulfonyl chloride (23 mg, 0.10 mmol) was added in one portion and the mixture was stirred at room temperature for 2 h. Brine (5 mL) was added and the reaction mixture was extracted with ethyl acetate (3×10 mL). The combined organic extracts were dried over anhydrous sodium sulfate and concentrated in vacuo. The resulting oil was subjected to chromatography on 25 g of silica gel and eluted with 9:1 hexane/ethyl acetate then 7:3 hexane/ethyl acetate to give the title compound as a white solid (23 mg, 70%): Mass spec. (EI): (M⁺) at 499.

### EXAMPLE 4

### (3S,6R)-Bicyclo[4.4.0]-1,4-diaza-3-(3-guanidinopropyl)-4-N-(4-tert-butylbenzenesulfonyl)-2,5-decanedione, trifluroacetate salt

### 4A) 1-[N^{γ}-(4-Methyltrityl)-N^{α}-(9-fluorenylmethoxy carbonyloxy)-L-ornithinyl]-D-2-piperidinecarboxylic acid, allyl ester

Example 1 pipecolic ester (500 mg, 1.86 mmol) was dissolved in 1:1 trifluroacetic acid/dichloromethane (8 mL) and stirred for 3 h. The reaction mixture was concentrated in vacuo and placed on a vacuum pump overnight. The resulting oil was dissolved in dimethylformamide (8 mL), cooled to 0° C. and diisopropylethylamine (0.97 mL, 5.58 mmol, Aldrich) was added. After stirring for 5 min, N^{γ}-(4-methyltrityl)-N^{α}-(9-fluorenylmethoxycarbonyloxy)-L-ornithine (1.36 g, 2.23 mmol, Novabiochem), N-hydroxybenzotriazole (398 mg, 2.60 mmol, Novabiochem) and 2-(1H-benzotriazole-1-yl)-1,1,3,3-tetramethyluronium tetrafluoroborate (835 mg, 2.60 mmol, Novabiochem) were added. The reaction mixture was stirred for 96 h, poured into ethyl acetate (125 mL) and washed with 10% hydrochloric acid (2×25 mL), saturated sodium bicarbonate solution (2×25 mL) and brine (25 mL). The organic layer was dried over anhydrous sodium sulfate and concentrated in vacuo. The resulting oil was subjected to chromatography on 75 g of silica gel and eluted with 3:1 hexane/ethyl acetate to give the title compound as a white solid (1.22 g, 86%).

### 4B) (3S,6R)-Bicyclo[4.4.0]-1,4-diaza-3-(3-N-(4-methyltrityl)aminopropyl)-2,5-decanedione

To a solution of Part 4A ester (500 mg, 0.66 mmol) in dichloromethane (132 mL) was added piperidine (3.26 mL, 33.0 mmol, Aldrich) and the reaction mixture was stirred for 3 h. The reaction mixture was concentrated in vacuo and the resulting oil was subjected to chromatography on 40 g of silica gel and eluted with 1:1 hexane/ethyl acetate to give the title compound as a white solid (288 mg, 91%).

### 4C) (3S,6R)-Bicyclo[4.4.0]-1,4-diaza-3-(3-N-(4-methyltrityl)aminopropyl)-4-N-(4-tert-butylbenzene-sulfonyl)-2,5-decanedione

To a solution of Part 4B diketopiperazine (150 mg, 0.31 mmol) in anhydrous tetrahydrofuran (5 mL, Aldrich) under a nitrogen atmosphere at 0° C. was added 1.0 M lithium bis(trimethylsilyl)amide in tetrahydrofuran (0.42 mL, 0.42 mmol, Aldrich) and the reaction mixture was stirred for 1 h. 4-tert-Butylbenzenesulfonyl chloride (109 mg, 0.47 mmol) was added in one portion and the mixture was stirred at room temperature for 2 h. Brine (10 mL) was added and the reaction mixture was extracted with ethyl acetate (3×20 mL). The combined organic extracts were dried over anhydrous sodium sulfate and concentrated in vacuo. The resulting oil was subjected to chromatography on 25 g of silica gel and eluted with 9:1 hexane/ethyl acetate then 7:3 hexane/ethyl acetate to give the title compound as a white solid (135 mg, 64%).

### 4D) (3S,6R)-Bicyclo[4.4.0]-1,4-diaza-3-(3-N,N′-(di-tert-butoxycarbonyl)guanidinopropyl)-4-N-(4-tert-butylbenzenesulfonyl)-2,5-decanedione

Part 4C diketopiperazine (296 mg, 0.44 mmol) was dissolved in 1% trifluoroacetic acid in dichloromethane (30 mL) and stirred for 30 min. The reaction mixture was concentrated in vacuo and the resulting oil was subjected to chromatography on 25 g of silica gel, eluted with 1:1 hexane/ethyl acetate then 4:1 dichloromethane/methanol and lyophilized to give (3S,6R)-bicyclo[4.4.0]-1,4-diaza-3-(3-aminopropyl)-4-N-(4-tert-butylbenzenesulfonyl)-2,5-decanedione, trifluoroacetate salt as a white solid.

To a solution of the above amine in dichloromethane (10 mL) was added triethylamine (0.061 mL, 0.44 mmol, Aldrich) and N,N′-di-tert-butoxy-N″-trifluoromethanesulfonyl guanidine (157 mg, 0.40 mmol, Journal of Organic Chemistry 63(12):3804-3805 (1998). After stirring for 12 h, the reaction mixture was poured into dichloromethane (50 mL) and washed with 1M aqueous sodium bisulfate (10 mL), 5% aqueous sodium bicarbonate (10 mL) and water (10 mL). The organic layer was dried over anhydrous sodium sulfate and concentrated in vacuo. The resulting oil was subjected to chromatography on 20 g of silica gel and eluted with 9:1 hexane/ethyl acetate then 1:1 hexane/ethyl acetate to give the title compound as a white solid (239 mg, 93%).

### 4E) (3S,6R)-Bicyclo[4.4.0]-1,4-diaza-3-(3-guanidinopropyl)-4-N-(4-tert-butylbenzenesulfonyl)-2,5-decanedione, trifluoroacetate salt

A solution of Part 4D diketopiperazine (100 mg, 0.16 mmol) was dissolved in 1:1 trifluroacetic acid/dichloromethane (2 mL), stirred for 1 h and the reaction mixture was concentrated in vacuo. The resulting oil was subjected to chromatography on 5 g of silica gel, eluted with 19:1 dichloromethane/methanol then 9:1 dichloromethane/methanol and lyophilized to give the title compound as a white solid (88 mg, 96%). Electrospray m.s.: (M+H⁺) at 464.5.

### EXAMPLE 5

### (3S,6R)-Bicyclo[4.4.0]-1,4-diaza-3-(4-guanidinobutyl)-4-N-(4-tert-butylsulfonyl)-2,5-decanedione, trifluroacetate salt

### 5A) 1-[N^{ε}-(4-Methyltrityl)-N^{α}-(9-fluorenylmethoxycarbonyloxy)-L-lysinyl]-D-2-piperidinecarboxylic acid, allyl ester

Example 1 pipecolic ester (253 g, 0.94 mmol) was dissolved in 1:1 trifluroacetic acid/dichloromethane (5 mL) and stirred for 2 h. The reaction mixture was concentrated in vacuo and placed on a vacuum pump overnight. The resulting oil was dissolved in dimethylformamide (5 mL), cooled to 0° C. and diisopropylethylamine (0.49 mL, 2.82 mmol, Aldrich) was added. After stirring for 5 min, N^{ε}-(4-methyltrityl)-N^{α}-(9-fluorenylmethoxycarbonyloxy)-L-lysine (706 mg, 1.13 mmol, Novabiochem), N-hydroxybenzotriazole (202 mg, 1.32 mmol, Novabiochem) and 2-(1H-benzotriazole-1-yl)-1,1,3,3-tetramethyluronium tetrafluoroborate (424 mg, 1.32 mmol, Novabiochem) were added. The reaction mixture was stirred for 72 h, poured into ethyl acetate (125 mL) and washed with 10% hydrochloric acid (2×25 mL), saturated sodium bicarbonate solution (2×25 mL) and brine (25 mL). The organic layer was dried over anhydrous sodium sulfate and concentrated in vacuo. The resulting oil was subjected to chromatography on 50 g of silica gel, eluted with 3:1 hexane/ethyl acetate to give the title compound as a white solid (605 mg, 83%).

### 5B) (3S,6R)-Bicyclo[4.4.0]-1,4-diaza-3-(4-N-(4-methyltrityl)butyl)-2,5-decanedione

To a solution of Part 5A ester (250 mg, 0.32 mmol) in dichloromethane (64 mL) was added piperidine (1.58 mL, 16.0 mmol, Aldrich) and the reaction mixture was stirred for 2 h. The reaction mixture was concentrated in vacuo and the resulting oil was subjected to chromatography on 25 g of silica gel and eluted with ethyl acetate to give the title compound as a white solid (149 mg, 94%).

### 5C) (3S,6R)-Bicyclo[4.4.0]-1,4-diaza-3-(4-N-(4-methyltrityl)butyl)-4-N-(4-tert-butylbenzenesulfonyl)-2,5-decanedione

To a solution of Part 5B diketopiperazine (50 mg, 0.10 mmol) in anhydrous tetrahydrofuran (1.5 mL, Aldrich) under a nitrogen atmosphere at 0° C. was added 1.0 M lithium bis(trimethylsilyl)amide in tetrahydrofuran (0.14 mL, 0.14 mmol, Aldrich) and the reaction mixture was stirred for 1 h. 4-tert-Butylbenzenesulfonyl chloride (35 mg, 0.15 mmol) was added in one portion and the mixture was stirred at room temperature for 2 h. Brine (5 mL) was added and the reaction mixture was extracted with ethyl acetate (3×10 mL). The combined organic extracts were dried over anhydrous sodium sulfate and concentrated in vacuo. The resulting oil was subjected to chromatography on 10 g of silica gel and eluted with 9:1 hexane/ethyl acetate then 7:3 hexane/ethyl acetate to give the title compound as a white solid (47 mg, 68%).

### 5D) (3S,6R)-Bicyclo[4.4.0]-1,4-diaza-3-(4-aminobutyl)-4-N-(4-tert-butylbenzenesulfonyl)-2,5-decanedione, trifluoroacetate salt

Part 5C diketopiperazine (57 mg, 0.082 mmol) was dissolved in 1% trifluoroacetic acid in dichloromethane (2 mL) and stirred for 15 min. The reaction mixture was concentrated in vacuo and the resulting oil was subjected to chromatography on 2 g of silica gel, eluted with 1:1 hexane/ethyl acetate then methanol and lyophilized to give the title compound as a white solid (40 mg, 89%).

### 5E) (3S,6R)-Bicyclo[4.4.0]-1,4-diaza-3-(4-N,N′-(di-tert-butoxycarbonyl)guanidinobutyl)-4-N-(4-tert-butylbenzenesulfonyl)-2,5-decanedione

To a solution of Part 5D amine (40 mg, 0.073 mmol) in dichloromethane (5 mL) was added triethylamine (0.011 mL, 0.082 mmol, Aldrich) and N,N′-di-tert-butoxy-N″-trifluoromethanesulfonyl guanidine (29 mg, 0.074 mmol, Journal of Organic Chemistry 63(12):3804-3805 (1998). After stirring for 12 h, the reaction mixture was poured into dichloromethane (25 mL) and washed with 1M aqueous sodium bisulfate (5 mL), 5% aqueous sodium bicarbonate (5 mL) and water (5 mL). The organic layer was dried over anhydrous sodium sulfate and concentrated in vacuo. The resulting oil was subjected to chromatography on 5 g of silica gel and eluted with 9:1 hexane/ethyl acetate then 1:1 hexane/ethyl acetate to give the title compound as a white solid (35 mg, 71%).

### 5F) (3S,6R)-Bicyclo[4.4.0]-1,4-diaza-3-(4-guanidinobutyl)-4-N-(4-tert-butylbenzenesulfonyl)-2,5-decanedione, trifluoroacetate salt

A solution of Part 5E diketopiperazine (35 mg, 0.052 mmol) was dissolved in 1:1 trifluroacetic acid/dichloromethane (1 mL), stirred for 1 h and the reaction mixture was concentrated in vacuo. The resulting oil was subjected to chromatography on 4 g of silica gel and eluted with 19:1 dichloromethane/methanol then 9:1 dichloromethane/methanol to give the title compound as a white solid (28 mg, 90%). Electrospray m.s.: (M+H⁺)@ 478.0.

### EXAMPLE 6

### (3S,6R)-Bicyclo[4.4.0]-1,4-diaza-3-(3-guanidinopropyl)-4-N-(3-methyl-8-quinolinesulfonyl)-2,5-decanedione, hydrochloride salt

### 6A) (3S,6R)-Bicyclo[4.4.0]-1,4-diaza-3-(3-N-(4-methyltrityl)aminopropyl)-4-N-(3-methyl-8-quinolinesulfonyl)-2,5-decanedione

To a solution of Example 4 Part B diketopiperazine (350 mg, 0.73 mmol) in anhydrous tetrahydrofuran (12 mL, Aldrich) under a nitrogen atmosphere at 0° C. was added 1.0 M lithium bis(trimethylsilyl)amide in tetrahydrofuran (0.88 mL, 0.88 mmol, Aldrich) and the reaction mixture was stirred for 1 h. 3-Methyl-8-quinolinesulfonyl chloride (168 mg, 0.69 mmol) was added in one portion and the mixture was stirred at room temperature for 2 h. Brine (15 mL) was added and the reaction mixture was extracted with ethyl acetate (3×25 mL). The combined organic extracts were dried over anhydrous sodium sulfate and concentrated in vacuo. The resulting oil was subjected to chromatography on 35 g of silica gel and eluted with 1:1 hexane/ethyl acetate to give the title compound as a white solid (304 mg, 64%).

### 6B) (3S,6R)-Bicyclo[4.4.0]-1,4-diaza-3-(3-amino-propyl)-4-N-(3-methyl-8-quinolinesulfonyl)-2,5-decanedione, trifluoroacetate salt

Part 6A diketopiperazine (304 mg, 0.44 mmol) was dissolved in 1% trifluoroacetic acid in dichloromethane (30 mL) and stirred for 30 min. The reaction mixture was concentrated in vacuo and the resulting oil was subjected to chromatography on 25 g of silica gel, eluted with 1:1 hexane/ethyl acetate then 4:1 dichloromethane/methanol and lyophilized to give the title compound as a white solid (240 mg, 100%).

### 6C) (3S,6R)-Bicyclo[4.4.0]-1,4-diaza-3-(3-N,N′-(di-tertbutoxycarbonyl)guanidinopropyl)-4-N-(3-methyl-8-quinolinesulfonyl)-2,5-decanedione

To a solution of Part 6B amine (240 mg, 0.44 mmol) in dichloromethane (10 mL) was added triethylamine (0.12 mL, 0.88 mmol, Aldrich) and N,N′-di-tert-butoxy-N″-trifluoromethanesulfonyl guanidine (164 mg, 0.42 mmol, Journal of Organic Chemistry 63(12):3804-3805 (1998). After stirring for 12 h, the reaction mixture was poured into dichloromethane (50 mL) and washed with 1M aqueous sodium bisulfate (10 mL), 5% aqueous sodium bicarbonate (10 mL) and water (10 mL). The organic layer was dried over anhydrous sodium sulfate and concentrated in vacuo. The resulting oil was subjected to chromatography on 25 g of silica gel and eluted with 1:1 hexane/ethyl acetate to give the title compound as a white solid (216 mg, 73%).

### 6D) (3S,6R)-Bicyclo[4.4.0]-1,4-diaza-3-(3-guanidinopropyl)-4-N-(3-methyl-8-quinolinesulfonyl)-2,5-decanedione, hydrochloride salt

A solution of Part 6C diketopiperazine (10 mg, 0.015 mmol) was dissolved in 3N hydrochloric acid in ethyl acetate (0.27 mL), stirred for 1 h, the reaction mixture was concentrated in vacuo and lyophilized to give the title compound as a white solid (7 mg, 88%). Electrospray m.s. : (M+H⁺)@ 473.5.

### EXAMPLE 7

### (3S,6R,8R)-Bicyclo[4.4.0]-1,4-diaza-3-(3-guanidinopropyl)-4-N-(3-methyl-8-quinolinesulfonyl)-8-methyl-2,5-decanedione, hydrochloride salt

### 7A) (2R, 4R) -1-[N^{γ}-(4-Methyltrityl)-N^{α}-(9-fluorenylmethoxycarbonyloxy)-L-ornithinyl]-4-methyl-2-piperidinecarboxylic acid, allyl ester

Example 2 pipecolic ester (406 mg, 1.43 mmol) was dissolved in 1:1 trifluroacetic acid/dichloromethane (7 mL) and stirred for 2 h. The reaction mixture was concentrated in vacuo and placed on a vacuum pump overnight. The resulting oil was dissolved in dimethylformamide (7 mL), cooled to 0° C. and diisopropylethylamine (0.75 mL, 4.29 mmol, Aldrich) was added. After stirring for 5 min, N^{γ}-(4-methyltrityl)-N^{α}-(9-fluorenylmethoxycarbonyloxy)-L-ornithine (1.05 g, 1.72 mmol, Novabiochem), N-hydroxybenzotriazole (306 mg, 2.00 mmol, Novabiochem) and 2-(1H-benzotriazole-1-yl)-1,1,3,3-tetramethyluronium tetrafluoroborate (642 mg, 2.00 mmol, Novabiochem) were added. The reaction mixture was stirred for 72 h, poured into ethyl acetate (125 mL) and washed with 10% hydrochloric acid (2×25 mL), saturated sodium bicarbonate solution (2×25 mL) and brine (25 mL). The organic layer was dried over anhydrous sodium sulfate and concentrated in vacuo. The resulting oil was subjected to chromatography on 75 g of silica gel and eluted with 3:1 hexane/ethyl acetate to give the title compound as a white solid (1.05 g, 95%).

### 7B) (3S,6R,8R)-Bicyclo[4.4.0]-1,4-diaza-3-(3-N-(4-methyltrityl)aminopropyl)-8-methyl-2,5-decanedione

To a solution of Part 7A ester (958 mg, 1.23 mmol) in dichloromethane (246 mL) was added piperidine (6.1 mL, 61.7 mmol, Aldrich) and the reaction mixture was stirred for 4 h. The reaction mixture was concentrated in vacuo and the resulting oil was subjected to chromatography on 65 g of silica gel and eluted with 4:1 hexane/ethyl acetate then 1:1 hexane/ethyl acetate to give the title compound as a white solid (520 mg, 96%).

### 7C) (3S,6R,8R)-Bicyclo[4.4.0]-1,4-diaza-3-(3-N-(4-methyltrityl)aminopropyl)-4-N-(3-methyl-8-quinoline-sulfonyl)-8-methyl-2,5-decanedione

To a solution of Part 7B diketopiperazine (250 mg, 0.50 mmol) in anhydrous tetrahydrofuran (7 mL, Aldrich) under a nitrogen atmosphere at 0° C. was added 1.0 M lithium bis(trimethylsilyl)amide in tetrahydrofuran (0.50 mL, 0.50 mmol, Aldrich) and the reaction mixture was stirred for 1 h. 3-Methyl-8-quinolinesulfonyl chloride (97 mg, 0.40 mmol) was added in one portion and the mixture was stirred at room temperature for 2 h. Brine (7 mL) was added and the reaction mixture was extracted with ethyl acetate (3×20 mL). The combined organic extracts were dried over anhydrous sodium sulfate and concentrated in vacuo. The resulting oil was subjected to chromatography on 25 g of silica gel and eluted with 3:1 hexane/ethyl acetate then 3:2 hexane/ethyl acetate to give the title compound as a white solid (177 mg, 63%).

### 7D) (3S,6R,8R)-Bicyclo[4.4.0]-1,4-diaza-3-(3-aminopropyl)-4-N-(3-methyl-8-quinolinesulfonyl)-8-methyl-2,5-decanedione, trifluoroacetate salt

Part 7C diketopiperazine (264 mg, 0.38 mmol) was dissolved in 1% trifluoroacetic acid in dichloromethane (26 mL) and stirred for 30 min. The reaction mixture was concentrated in vacuo and the resulting oil was subjected to chromatography on 20 g of silica gel, eluted with 1:1 hexane/ethyl acetate then 19:1 dichloromethane/methanol and lyophilized to give the title compound as a white solid (206 mg, 100%).

### 7E) (3S,6R,8R)-Bicyclo[4.4.0]-1,4-diaza-3-(3-N,N′-(di-tert-butoxycarbonyl)guanidinopropyl)-4-N-(3-methyl-8-quinolinesulfonyl)-8-methyl-2,5-decanedione

To a solution of Part 7D amine (206 mg, 0.38 mmol) in dichloromethane (8 mL) was added triethylamine (0.053 mL, 0.38 mmol, Aldrich) and N,N′-di-tert-butoxy-N″-trifluoromethanesulfonyl guanidine (82 mg, 0.34 mmol, Journal of Organic Chemistry 63(12):3804-3805 (1998). After stirring for 12 h, the reaction mixture was poured into dichloromethane (50 mL) and washed with 1M aqueous sodium bisulfate (10 mL), 5% aqueous sodium bicarbonate (10 mL) and water (10 mL). The organic layer was dried over anhydrous sodium sulfate and concentrated in vacuo. The resulting oil was subjected to chromatography on 15 g of silica gel and eluted with 1:1 hexane/ethyl acetate to give the title compound as a white solid (140 mg, 60%).

### 7F) (3S,6R,8R)-Bicyclo[4.4.0]-1,4-diaza-3-(3-guanidinopropyl)-4-N-(3-methyl-8-quinolinesulfonyl)-8-methyl-2,5-decanedione, hydrochloride salt

A solution of Part 7E diketopiperazine (10 mg, 0.15 mmol) was dissolved in 3N hydrochloric acid in ethyl acetate (0.27 mL), stirred for 1 h, the reaction mixture was concentrated in vacuo and lyophilized to give the title compound as a white solid (7.5 mg, 94%). Electrospray m.s. : (M+H⁺) at 487.5.

### EXAMPLE 8

### (3S,6R,8R)-Bicyclo[4.4.0]-1,4-diaza-3-(3-guanidinopropyl)-4-N-(1,2,3,4-tetrahydro-3-methyl-8-quinolinesulfonyl)-8-methyl-2,5-decanedione, hydrochoride salt

### 8A) (2R,4R)-1-[N^{γ}-(4-Methoxy-2,3,6-trimethyl-benzenesulfonyl)-N^{α}-(tert-butoxycarbonyl)-L-arginyl]-4-methyl-2-piperidinecarboxylic acid

Example 2 pipecolic ester (375 mg, 1.32 mmol) was dissolved in 1:1 trifluroacetic acid/dichloromethane (8 mL) and stirred for 2 h. The reaction mixture was concentrated in vacuo and placed on a vacuum pump overnight. The resulting oil was dissolved in dimethylformamide (8 mL), cooled to 0° C. and diisopropylethylamine (1.15 mL, 6.6 mmol, Aldrich) was added. After stirring for 5 min, N^{γ}-(4-methoxy-2,3,6-trimethylbenznesulfonyl)-N^{α}-(tert-butoxycarbonyl)-L-arginine (769 mg, 1.58 mmol, Novabiochem), N-hydroxybenzotriazole (283 mg, 1.85 mmol, Novabiochem) and 2-(1H-benzotriazole-1-yl)-1,1,3,3-tetramethyluronium (594 mg, 1.85 mmol, Novabiochem) were added. The reaction mixture was stirred for 18 h, poured into ethyl acetate (75 mL) and washed with 10% citric acid (2×10 mL), saturated sodium bicarbonate solution (2×10 mL) and brine (10 mL). The organic layer was dried over anhydrous sodium sulfate and concentrated in vacuo to give (2R,4R)-1-[N^{γ}-(4-methoxy-2,3,6-trimethylbenzenesulfonyl)-N^{α}-(tert-butoxycarbonyl)-L-arginyl]-4-methyl-2-piperidinecarboxylic acid, allyl ester as a white foamy solid.

The peptide from above was dissolved in 1:1 trifluoroacetic acid/dichloromethane (8 mL) and stirred for 5 min. The reaction mixture was concentrated in vacuo and placed on a vacuum pump for 5 min. The resulting oil was dissolved in dichloromethane (20 mL) and triethylamine (1.8 mL, 13.2 mmol, Aldrich) and 3-methyl-8-quinolinesulfonyl chloride (319 mg, 1.32 mmol) were added. After stirring for 1 h, the reaction mixture was poured into dichloromethane (50 mL) and washed with water (15 mL) and brine (15 mL). The organic layer was dried over anhydrous sodium sulfate and concentrated in vacuo to give (2R,4R)-1-[N^{γ}-(4-methoxy-2,3,6-trimethylbenzenesulfonyl)-N^{α}-(3-methyl-8-quinolinesulfonyl)-L-arginyl]-4-methyl-2-piperidinecarboxylic acid, allyl ester as a pale yellow foamy solid.

The peptide from above was dissolved in absolute ethanol (14 mL) and 1N aqueous sodium hydroxide (3.6 mL). After stirring for 21 h, the reaction mixture was adjusted to pH 7 with 1N hydrochloric acid and concentrated in vacuo. The resulting residue was dissolved in 1:1 ethyl acetate/water (20 mL), the solution was adjusted to pH 11 with 1N sodium hydroxide and extracted with ethyl acetate (2×30 mL). The aqueous layer was adjusted to pH 2 with 1N hydrochloric acid and extracted with chloroform (3×50 mL). The combined chloroform extracts were dried over anhydrous sodium sulfate and concentrated in vacuo to give the title compound as a white foamy solid (829 mg, 88% over 3 steps).

### 8B) (2R,4R)-1-[N^{γ}-(4-Methoxy-2,3,6-trimethylbenzenesulfonyl)-N^{α}-(1,2,3,4-tetrahydro-3-methyl-8-quinolinesulfonyl)-L-arginyl]-4-methyl-2-piperidinecarboxylic acid

A suspension of Part 8A acid (100 mg, 0.14 mmol) and 10% palladium on carbon (28 mg, Aldrich) in 95% ethanol (2 mL) and 1N hydrochloric acid (0.12 mL) was heated in a 15 mL sealed tube under a hydrogen atmosphere at 75°-80° C. for 65 h. The mixture was cooled to room temperature, filtered and concentrated in vacuo. The resulting oil was subjected to chromatography on 15 g of silica gel and eluted with ethyl acetate then 4:1 dichloromethane/methanol to give the title compound as a white solid (58 mg, 58%).

### 8C) (3S,6R,8R)-bicyclo[4.4.0]-1,4-diaza-3-(3-(N^{γ}-4-methoxy-2,3,6-rimethylbenzenesulfonyl)guanidinopropyl)-4-N-(3-methyl-1,2,3,4-tetrahydro-8-quinolinesulfonyl)-8-methyl-2,5-decanedione

To a solution of Part 8B acid (58 mg, 0.080 mmol) in dichloromethane (16 mL) was added N-hydroxybenzotriazole (12 mg, 0.080 mmol, Novabiochem), 2-(1H-benzotriazole-1-yl)-1,1,3,3-tetramethyluronium tetrafluoroborate (26 mg, 0.080 mmol, Novabiochem) and diisopropylethylamine (0.014 mL, 0.080 mmol, Aldrich). The reaction mixture was stirred for 3 h, poured into ethyl acetate (30 mL) and washed with saturated sodium bicarbonate solution (10 mL) and brine (10 mL). The organic layer was dried over anhydrous sodium sulfate and concentrated in vacuo. The resulting oil was subjected to chromatography on 6 g of silica gel, eluted with 1:4 hexane/ethyl acetate and lyophilized to give the title compound as a white solid (51 mg, 91%).

### 8D) (3S,6R,8R)-Bicyclo[4.4.0]-1,4-diaza-3-(3-guanidinopropyl)-4-N-(1,2,3,4-tetrahydro-3-methyl-8-quinolinesulfonyl)-8-methyl-2,5-decanedione, hydrochoride salt

Part 8C diketopiperazine (36 mg, 0.051 mmol) was dissolved in 1:1 trifluoroacetic acid/dichloromethane (4 mL), stirred for 20 h and concentrated in vacuo. The resulting oil was dissolved in 3N hydrochloric acid in ethyl acetate (4 mL), stirred for 1 h and concentrated in vacuo. The resulting oil was subjected to chromatography on 2 g of silica gel, eluted with ethyl acetate then methanol and lyophilized to give the title compound as a white solid (20 mg, 74%). Electrospray m.s.: (M+H⁺) at 491.5.

### EXAMPLE 9

### Biological Assays of Cycloargatroban (Formula I) where R¹, R² and R⁴ are hydrogen, R³ is Me=CH₃, R⁵ is 1,2,3,4-tetrahydro-3-methyl-8-quinolinesulfonyl and R⁶ is 3-guanidinopropyl

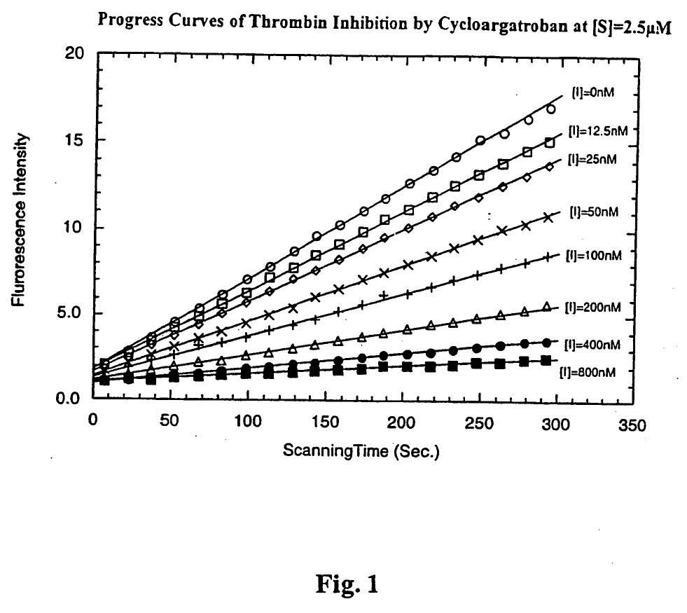

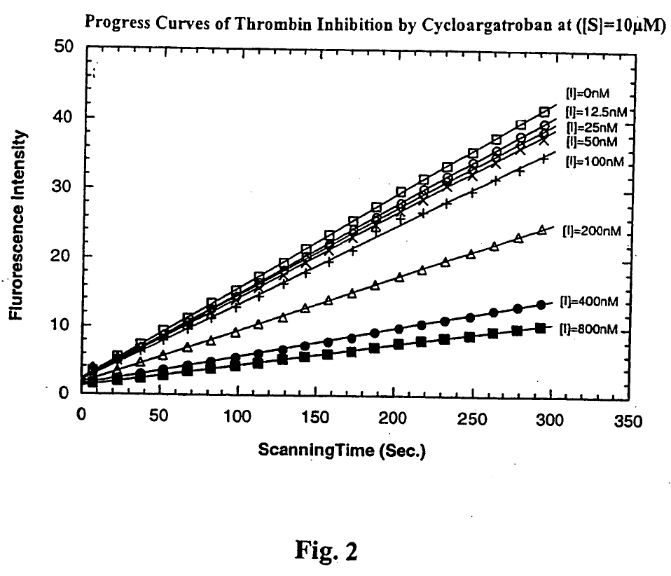

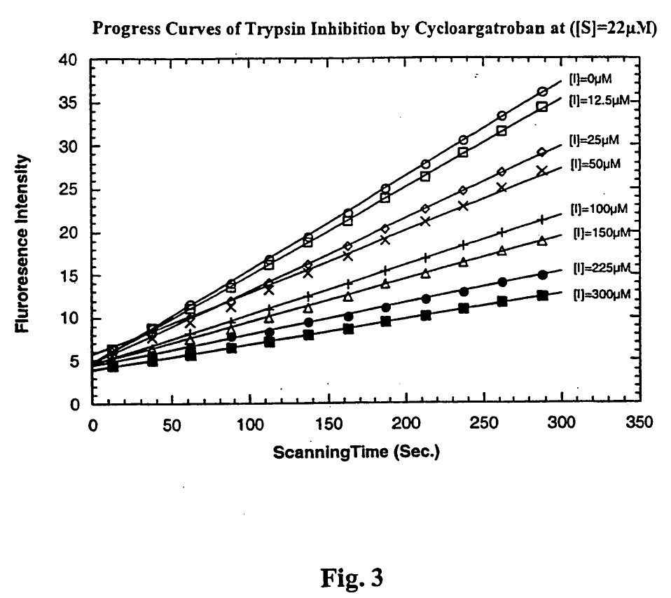

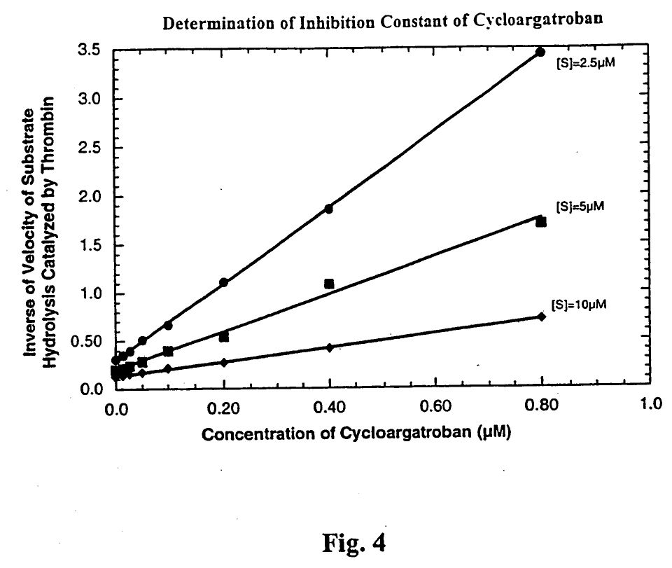

The activity and selectivity of the present invention can be identified by determination of the inhibition constant (Ki) for serine proteases such thrombin and trypsin and fibrinolytic enzymes such as urokinase, plasmin and tissue plasminogen activator (tPA). All of enzymes are purchased from Sigma. The general assay conditions are as follows. The fluorogenic substrates are dissolved in DMSO and diluted using assay buffer containing 50 mM Tris.HCl (pH 7.8 at 25° C.), 0.1 M NaCl and 0.1% polyethylene glycol 8000 (PEG 8000). The fluorogenic substrates are Tos-Gly-Pro-Arg-AMC (Sigma, Km=4.0 μM at 25° C., pH7.8)(Yudu Cheng et al., Biochemistry, 1996, 35: 13021-13029) for thrombin, Bz-Arg-AMC.HCl (Bachem, Km=59±2 μM at 25° C., pH8.0) for trypsin, N-Cbz-Gly-Gly-Arg-AMC (Sigma, Kₘ=400 μM at 24° C. and pH 7.5) for urokinase, D-Ala-Leu-Lys-AMC (Sigma, Kₘ=620 μM at 25° C. and pH 8.0) for plasmin and Boc-L-(p-F) FPR-ANSNH-C₂H₅ (Haematologic Technologies Inc., Kₘ=71 μM at 25° C. and pH 7.4) for tPA. The assays were conducted using Hitachi F2500 spectrophotometer under ambient temperature and at the excitation and emission wavelengths of 383 nm and 455 nm, respectively. The typical progressive data of the enzymatic assays are shown in FIGS. 1-3, and the determination of inhibition constant (Ki) is shown in FIG. 4. The assay results, in comparison to argatroban, an anticoagulant currently in clinic use and with the chemical structure XIII, are shown in Table I.

The results demonstrate that the cycloargatroban derived from the cyclization of backbone of argatroban are featured by: (1) Retaining high thrombin inhibition activity (2.1-fold lower than argatroban); (2) Achieving high selectivity for thrombin over trypsin (12-fold higher than argatroban); (3) Retaining no significant inhibition for fibrinolytic enzymes (similar to argatroban); (4) Retaining the diversity in side chains (similar to argatroban).

TABLE I
Assay Results of comparision of Argatroban (see chemical structure
XIII below) and Cycloargatroban (Formula I) where R¹, R² and
R⁴ are hydrogen, R³ is Me═CH₃, R⁵ is 1,2,3,4-
tetrahydro-3-methyl-8-quinolinesulfonyl and R⁶ is 3-guanidinopropyl

| | |
|---|---|
| | XIII |
| 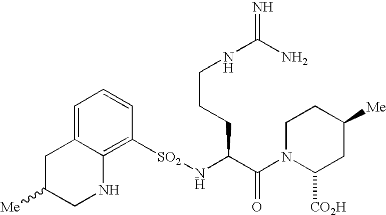 | |

| | | | Cycloargatroban vs. |
|---|---|---|---|
| Activity | Cycloargatroban | Argatroba | Argatroban* |
| Kᵢ(Thrombin) | 40 nM | 19 nM | −2.1 fold |
| Kᵢ(Trypsin) | 126 μM | 5 μM | +12 fold |
| Kᵢ(Urokinase) | 295 μM | 999 μM | −0.14 fold |
| Kᵢ(Plasmin) | 528 μM | 372 μM | −0.67 fold |
| Kᵢ(tPA) | 2021 μM | 777 μM | +1.2 fold |

*Cycloargatroban [Kᵢ (Enzyme)/Kᵢ (Thrombin)] vs. Argatroban [Kᵢ (Enzyme)/Kᵢ (Thrombin)]

### EXAMPLE 10

### Ex vivo Coagulation Assay

The ex vivo anticoagulant effects of NPI999 in comparison with argatroban, a reference anticoagulant currently in clinical use with the following chemical structure: 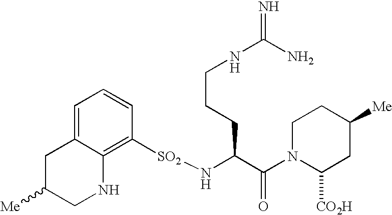
were determined by measuring the prolongation of the activated partial thromboplastin time (APTT) over a broad concentration range of each added thrombin inhibitor, using pooled normal human plasma. Frozen-pooled human plasma was obtained from Sigma. Measurement of APTT was made using the ELECTRA™ 800 automated coagulometer (Medical Laboratory Automation Inc.) using the automated APTT reagent (SIGMA) as the initiator of clotting according to the manufacture's instructions. The assay was conducted by making a series of dilution of the reference and test compounds in rapidly thawed plasma (compound: plasma=0.1 ml:0.9 ml) followed by adding the mixed solution to the wells of the assay carousel. Tris buffers (pH 7.8 at 25° C.) were used through the entire assay.

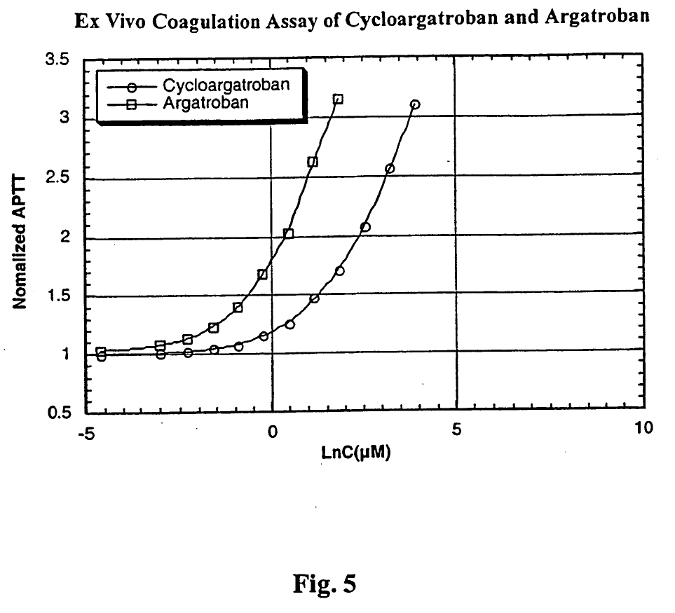

FIG. 5 depicts the effect of NPI999 (open circle) and argatroban (open square) on the activated partial thromboplastin time (APTT) of normal citrated human plasma. As shown in FIG. 5, both compounds prolonged the APTT in a dose dependent manner. This demonstrates the deactivation of coagulating enzymes presented in the human plasma. It is to be noted that APTT measures the overall anticoagulant effects of a compound against the clotting enzymes such as thrombin, plasmin, urokinase, tissue plasminogen activator (tPA) and serine protease such as trypsin, factor X etc. Therefore, the less strong effect of cycloargatroban (formula I) than argatroban on APTT may be attributed to higher selectivity of cycloargatroban (formula I) to the clotting and serine protease than argatroban.

FIG. 5 depicts the effect of NPI1999 (open circle) and argatroban (open square) on the activated partial thromboplastin time (APTT) of normal citrated human plasma. As shown in FIG. 5, both compounds prolonged the APTT in a dose dependent manner. This demonstrates the deactivation of coagulating enzymes presented in the human plasma. It is to be noted that APTT measures the overall anticoagulant effects of a compound against the clotting enzymes such as thrombin, plasmin, urokinase, tissue plasminogen activator (tPA) and serine protease such as trypsin, factor X etc. Therefore, the less strong effect of cycloargatroban (formula I) than argatroban on APTT may be attributed to higher selectivity of cycloargatroban (formula I) to the clotting and serine protease than argatroban.

### EXAMPLE 11

### Assay Results of Cycloargatroban Derivatives

TABLE II

| | |
|---|---|
| | I |
| 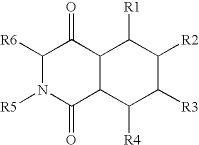 | |

| No. | Name of Analog | Chemical Structure | Ki(Thrombin) |
|---|---|---|---|
| 1 | Lead Compound | R1=R2=R4=H, R3=Methyl, | 0.020-0.040 μM |
| | (Formula I) | R5=1,2,3,4-tetrahydro-3- | |
| | | methyl-8- | |
| | | quinolinesulfonyl, | |
| | | R6=3-guanidinopropyl | |
| 2 | Ehtyl Analogue | R1=R2=R4=H, R3=Ethyl, | 0.061 μM |
| | | R5=1,2,3,4-tetrahydro-3- | |
| | | methyl-8- | |
| | | quinolinesulfonyl, | |
| | | R6=3-guanidinopropyl | |
| 3 | Phenyl Analog | R1=R2=R4=H, R3=Phenyl, | 0.91 μM |
| | | R5=1,2,3,4-tetrahydro-3- | |
| | | methyl-8- | |
| | | quinolinesulfonyl, | |
| | | R6=3-guanidinopropyl | |
| 4 | t-Butyl Analog | R1=R2=R4=H, R3=t-Butyl, | 1.58 μM |
| | | R5=1,2,3,4-tetrahydro-3- | |
| | | methyl-8- | |
| | | quinolinesulfonyl, | |
| | | R6=3-guanidinopropyl | |
| 5 | Arginine Mimic | R1=R2=R4=H, R3=Methyl, | 0.059 μM |
| | | R5=1,2,3,4-tetrahydro-3- | |
| | | methyl-8- | |
| | | quinolinesulfonyl, | |
| | | R6=4-Amidinopheyalanine | |

The above-described lead compound (Compound No. 1) and four analogs were chemically synthesized and biologically tested. The chemical synthesis method is described in examples 1-8. The biological activity test (Ki-Thrombin) method is described in example 9. The results indicate that the lead compound and the four analogues specifically inhibit thrombin.

### EXAMPLE 12

### In Vivo Test of NPI1999 (Compound No. 1)

In vivo tests of NPI1999 were performed using ICR mice (Institute for Cancer Research), and SD (Sprague Dawley) rats to measure the acute toxicity, effect on blood coagulation system, Deep Vein Thrombosis (DVT) and Acute Myocardial infarction (AMI) by using FDA-approved thrombin inhibitor (argatroban™) as reference drug. All of the test results indicated that NPI1999 (Compound 1) is not only effective in vitro but also effective in vivo as shown by its ability to reduce and/or eliminate the major symptoms caused by the coagulation mediated by thrombin. In some assays, the efficacy of NPI1999 was higher than that of argatroban™. This advantage is significant since it enables the use of lower dosage of NPI1999 compared to (argatroban™) and may lead to less bleeding side effects. The results are summarized as follows:

### Acute Toxicity

1) Determination of LD₅₀ (Tables III and IV): ICR mice, 18 ˜20 g, were divided into five groups, with 10 animals in each group (half male and half female). The ratio of doses between groups was 1:0.85. The drug was administered intravenously once, and the mice were observed for 2 weeks. LD₅₀ and the 95% confidence interval, calculated according to the Bliss method, were 10.91 mg/kg and 10.00 mg/kg˜11.89 mg/kg respectively.

TABLE III
The death distribution of mice after intravenous injection of NPI1999

| | | | | | | | | | 4-13 days | |
|---|---|---|---|---|---|---|---|---|---|---|
| | day of | | 1 day after | | 2 days after | | 3 days after | | after | |
| | administration | | administration | | administration | | administration | | administration | |
| Dose | | Number of | | Number of | | Number of | | Number of | | Number of |
| (mg/kg) | sex | death | sex | death | sex | death | sex | death | sex | death |
| 7.83 | ♂ | 0 | ♂ | 0 | ♂ | 0 | ♂ | 0 | ♂ | 0 |
| | ♀ | 0 | ♀ | 0 | ♀ | 0 | ♀ | 0 | ♀ | 0 |
| 9.21 | ♂ | 1 | ♂ | 0 | ♂ | 0 | ♂ | 0 | ♂ | 0 |
| | ♀ | 2 | ♀ | 0 | ♀ | 0 | ♀ | 0 | ♀ | 0 |
| 10.84 | ♂ | 2 | ♂ | 0 | ♂ | 0 | ♂ | 0 | ♂ | 0 |
| | ♀ | 3 | ♀ | 0 | ♀ | 0 | ♀ | 0 | ♀ | 0 |
| 12.75 | ♂ | 4 | ♂ | 0 | ♂ | 0 | ♂ | 0 | ♂ | 0 |
| | ♀ | 4 | ♀ | 0 | ♀ | 0 | ♀ | 0 | ♀ | 0 |
| 15.00 | ♂ | 5 | ♂ | 0 | ♂ | 0 | ♂ | 0 | ♂ | 0 |
| | ♀ | 4 | ♀ | 0 | ♀ | 0 | ♀ | 0 | ♀ | 0 |

2) Determination of the maximal dose (Table V): ICR mice(n=20) were administered by intragastric gavage (i.g.) twice with a dose of 1.43 g/kg with an interval of 8 h between administrations. The dose is based on maximal dissolution rate and maximal administered volume of the drug. No mortality was observed for up to one week treatment. The maximal dose of i.g. was 2.86 g/kg/d.

TABLE IV
results of acute toxicity test in mice via intravenous
injection of NPI1999

| | Number | Number | | LD₅₀ and 95% |
|---|---|---|---|---|
| Dose | of | of | Death | confidence |
| (mg/kg) | Animals | deaths | rate (%) | interval |
| 7.83 | 10 | 0 | 0 | LD₅₀ = 10.91 mg/kg |
| 9.21 | 10 | 3 | 30 | 95% confidence |
| 10.84 | 10 | 5 | 50 | interval is |
| 12.75 | 10 | 8 | 80 | 10.00 mg/kg-11.89 mg/kg |
| 15.00 | 10 | 9 | 90 | |

TABLE V
The results of acute toxicity test in mice via oral
gavage of NPI1999

| | | | | Maximal |
|---|---|---|---|---|
| | | | | dose of oral |
| | Dose | Number of | Number of | gavage |
| Group | (g/kg) | Animals | Deaths | (g/kg) |
| Solvent | — | 20 | 0 | |
| Cycloargartroban | 1.43 | 20 | 0 | 2.86 |

### Effect on Blood Coagulation System

1) Determination of clotting time of mice blood (Table VI): ICR mice, half male and half female, 18-22 g, were divided into seven groups: control, positive control (Argatroban, 2.5 mg/kg), injected i.v. with 1 mg/kg (high dose) of NPI1999, injected i.v. with 0.5 mg/kg (middle dose) of NPI1999, injected i.v. with 0.25 mg/kg (low dose) of NPI1999, i.g. administration of 150 mg/kg (high dose) of NPI1999, and i.g. administration 75 mg/kg (low dose) of NPI1999 respectively. Blood samples were collected using the capillary method 0.5 h and 1.5 h after i.v. injections and 1 h and 3 h after i.g. and clotting time of whole blood was measured. The results indicate that at 0.5 h and 1.5 h after i.v. administration of NPI1999, the clotting time for the middle (0.5 mg/kg) and high (1 mg/kg) dose groups were significantly prolonged compared to controls. No significant difference in clotting time was observed after i.g. administration at low (75 mg/kg) and high (150 mg/kg) doses at either time point (1 h and 3 h).

TABLE VI
Effect of NPI1999 on CT

| | Dose and route | | | First time point | Second time point |
|---|---|---|---|---|---|
| | of | | Before | (0.5 h for i.v.) | (1.5 h for i.v.) |
| Group | administration | n | administration | (1 h for i.g.) | (3.0 h i.g.) |
| Control | i.v. | 18 | 118.39 ± 31.47 | 122.11 ± 30.56 | 123.72 ± 34.06 |
| Argatroban | 2.5 mg/kg i.v. | 18 | 133.28 ± 26.21 | 170.33 ± 28.34***### | 156.11 ± 32.08**### |
| NPI1999 | 0.25 mg/kg i.v. | 17 | 128.53 ± 27.14 | 140.02 ± 26.40## | 139.88 ± 30.40# |
| NPI1999 | 0.5 mg/kg i.v. | 17 | 125.53 ± 25.03 | 172.88 ± 34.26***### | 161.76 ± 40.10**### |
| NPI1999 | 1 mg/kg i.v. | 16 | 124.06 ± 28.96 | 165.12 ± 30.57***### | 163.62 ± 44.26**### |
| NPI1999 | 75 mg/kg i.g. | 18 | 126.11 ± 22.83 | 133.11 ± 29.00 | 119.39 ± 25.88 |
| NPI1999 | 150 mg/kg i.g. | 18 | 125.50 ± 23.67 | 128.06 ± 30.03 | 119.44 ± 26.01 |

**p < 0.01,
***p < 0.001 vs. control
#p < 0.05,
##p < 0.01,
###p < 0.001 vs. before administration

2) Determination of Clot Retraction Time (RT), Thrombin Time (TT), Prothrombin Time (PT), Activated Partial Prothromboplastin Time (APTT), Fibrillation (FIB) in rat plasma (Table VII to XI): SD rats, half male and half female, 200±20 g, were divided into five groups: control, positive control (Argatroban, 2.5 mg/kg), injected i.v. with 0.25 mg/kg (high dose) of NPI1999, injected i.v. with 0.125 mg/kg (low dose) of NPI1999, and i.g. administration of 75 mg/kg of NPI1999. Blood was taken at 0.5 h and 1.5 h after i.v. injection and at 1 h and 3 h after i.g. and plasma RT,TT,PT,APTT and FIB were determined. RT,TT,PT and APTT were significantly increased and FIB was reduced in i.v. groups at high dose (0.25 mg/kg) at the first time point (0.5 h). Only RT,TT,PT were increased at the second time point (1.5 h), whereas APTT and FIB were no significantly affected. At low dose (0.125 mg/kg) RT alone was increased at the first time point and none of the indexes were affected significantly at the second time point. None of the indexes was significantly affected either at the 1 h or 3 h time points in the i.g. group.

TABLE VII
Effect of NPI1999 on RT

| | | | | Second time |
|---|---|---|---|---|
| | Dose and route | | First time point | point |
| | of | | (0.5 h for i.v.) | (1.5 h for i.v.) |
| Group | administration | n | (1 h for i.g.) | (3.0 h i.g.) |
| Control | i.v. | 10 | 72.2 ± 10.2 | 64.1 ± 9.7 |
| Argatroban | 2.5 mg/kg | 11 | 91.2 ± 15.0** | 77.4 ± 10.6** |
| | i.v. | | | |
| NPI1999 | 0.125 mg/kg | 10 | 83.7 ± 9.7** | 60.4 ± 15.9 |
| | i.v. | | | |
| NPI1999 | 0.25 mg/kg | 10 | 107.6 ± 33.7** | 78.6 ± 16* |
| | i.v. | | | |
| NPI1999 | 75 mg/kg | 10 | 74.8 ± 16.6 | 77 ± 29.9 |
| | i.g. | | | |

*p < 0.05, **p < 0.01, ***p < 0.001 vs. control

TABLE VIII
Effect of NPI1999 on TT

| | Dose and | | | Second time |
|---|---|---|---|---|
| | route of | | First time point | point |
| | admin- | | (0.5 h for i.v.) | (1.5 h for i.v.) |
| Group | istration | n | (1 h for i.g.) | (3.0 h i.g.) |
| Control | i.v. | 10 | 46.6 ± 4.3 | 36.7 ± 8.1 |
| Argatroban | 2.5 mg/kg | 11 | 72.9 ± 20.1*** | 50.4 ± 12.3** |
| | i.v. | | | |
| NPI1999 | 0.125 mg/kg | 10 | 53.6 ± 11.1 | 39.2 ± 12.5 |
| | i.v. | | | |
| NPI1999 | 0.25 mg/kg | 10 | 62.1 ± 15.8** | 49.6 ± 10.9** |
| | i.v. | | | |
| NPI1999 | 75 mg/kg | 10 | 53.2 ± 10.0 | 42.5 ± 9.4 |
| | i.g. | | | |

*p < 0.05, **p < 0.01, ***p < 0.001 vs. control

TABLE IX
Effect of NPI1999 on PT

| | | | | Second time |
|---|---|---|---|---|
| | Dose and route | | First time point | point |
| | of | | (0.5 h for i.v.) | (1.5 h for i.v.) |
| Group | administration | n | (1 h for i.g.) | (3.0 h i.g.) |
| Control | i.v. | 10 | 26.5 ± 5.9 | 24.6 ± 4.5 |
| Argatroban | 2.5 mg/kg i.v. | 11 | 37.9 ± 8.6** | 29.5 ± 8.5 |
| NPI1999 | 0.125 mg/kg i.v. | 10 | 25.3 ± 5.8 | 25.0 ± 7.1 |
| NPI1999 | 0.25 mg/kg i.v. | 10 | 43.1 ± 17.8* | 34.6 ± 10.1* |
| NPI1999 | 75 mg/kg i.g. | 10 | 26.6 ± 5.5 | 26.6 ± 5.7 |

*p < 0.05, **p < 0.01, ***p < 0.001 vs. control

TABLE X
Effect of NPI1999 on APTT

| | | | | Second time |
|---|---|---|---|---|
| | Dose and route | | First time point | point |
| | of | | (0.5 h for i.v.) | (1.5 h for i.v.) |
| Group | administration | n | (1 h for i.g.) | (3.0 h i.g.) |
| Control | i.v. | 10 | 27.3 ± 7.6 | 28.3 ± 4.4 |
| Argatroban | 2.5 mg/kg i.v. | 11 | 37.9 ± 10.2* | 30.1 ± 6.5 |
| NPI1999 | 0.125 mg/kg i.v. | 10 | 28.0 ± 4.0 | 28.1 ± 4.6 |
| NPI1999 | 0.25 mg/kg i.v. | 10 | 46.7 ± 15.0** | 34.1 ± 10.3 |
| NPI1999 | 75 mg/kg i.g. | 10 | 30.2 ± 6.9 | 25.7 ± 4.0 |

*p < 0.05, **p < 0.01, ***p < 0.001 vs. control

TABLE XI
Effect of NPI1999 on FIB

| | | | | Second time |
|---|---|---|---|---|
| | Dose and route | | First time point | point |
| | of | | (0.5 h for i.v.) | (1.5 h for i.v.) |
| Group | administration | n | (1 h for i.g.) | (3.0 h i.g.) |
| Control | i.v. | 10 | 4.3 ± 0.67 | 6.0 ± 3.1 |
| Argatroban | 2.5 mg/kg i.v. | 11 | 2.70 ± 0.8*** | 4.0 ± 0.9 |
| NPI1999 | 0.125 mg/kg i.v | 10 | 4.2 ± 0.9 | 4.5 ± 1.2 |
| NPI1999 | 0.25 mg/kg i.v. | 10 | 2.09 ± 0.5*** | 2.7 ± 0.8 |
| NPI1999 | 75 mg/kg i.g. | 10 | 4.4 ± 0.7 | 4.5 ± 0.6 |

*p < 0.05, **p < 0.01, ***p < 0.001 vs. control

### Determination of the Weight of Deep Vein Thrombosis in Rats

SD rats, 220±20 g, were divided into six groups (n=10, half male and half female): model, positive control (Argatroban, 1.25 mg/kg), injected i.v. with 0.5 mg/kg (high dose) of NPI1999, injected i.v. with 0.25 mg/kg (middle dose) of NPI1999, injected i.v. with 0.125 mg/kg (low dose) of NPI1999, and a group that was administered intra-duodenum at a dose of 75 mg/kg. The drug was administered immediately after operation and, 4 hours later, the thrombi were collected and weighted. The results (Table XII) show that the thrombi weights were decreased in i.v. groups but no obvious effect was observed in the group administered intra-duodenum.

TABLE XII
Effect of NPI1999 on thrombi of inferior vena cava in
rats (x ± s, n = 10)

| | Dose and route | | |
|---|---|---|---|
| | of | Thrombi (mg) | |
| Group | administration | Wet weight | Dry weight |
| Model | — | 44.08 ± 20.74 | 13.87 ± 5.62 |
| Argatroban | 1.25 mg/kg i.v. | 18.04 ± 8.99** | 6.26 ± 3.72** |
| NPI1999 | 0.50 mg/kg i.v. | 18.85 ± 8.61** | 5.81 ± 3.25** |
| NPI1999 | 0.25 mg/kg i.v. | 19.63 ± 12.02** | 6.70 ± 4.34** |
| NPI1999 | 0.125 mg/kg i.v. | 19.84 ± 12.17** | 7.62 ± 6.18* |
| NPI1999 | 75 mg/kg | 32.02 ± 19.34 | 11.66 ± 4.23 |
| | intraduodenum | | |

*p < 0.05, **p < 0.01 vs. model

### Acute Myocardial infarction

SD rats, 220±20 g, were divided into six groups (n=10, half male and half female) : control (sham-operation), model, positive control (Argatroban, o.125 mg/kg), injected i.v. with 0.25 mg/kg (high dose) of NPI1999, injected i.v. with 0.125 mg/kg (low dose) of NPI1999, and a group that was administered intra-duodenum at a dose of 75 mg/kg. The drugs were administered 8 to 10 min after the coronary artery was ligated. The electrocardiogram (ECG) was observed (see results in Tables XIV and XV) for 6 h and 8 h after i.v. and intra-duodenum administration respectively, the heart was then excised and stained and blood was collected. The results indicate (Table XIII) that infarction area and serum LDH and CK were significantly reduced at high dose (0.25 mg/kg) and low dose (0.125 mg/kg) in the i.v. group, whereas no significant effects were observed in intra-duodenum administration group.

TABLE XIII
Effect of NPI1999 on infarction area caused by coronary artery ligation, LDH and CK in

| | Dose and route | | | |
|---|---|---|---|---|
| | of | Infarction | | |
| Group | administration | Area (%) | LDH (u/ml) | CK (u/ml) |
| Model | — | 33.56 ± 4.08 | 6663.5 ± 394.1 | 74.86 ± 21.95 |
| Sham-operation | — | 10.80 ± 3.08*** | 5841.9 ± 508.0*** | 50.29 ± 15.11 |
| NPI1999 | 0.25 mg/kg i.v. | 25.66 ± 7.32* | 6125.7 ± 385.5** | 56.52 ± 14.48* |
| NPI1999 | 0.125 mg/kg i.v. | 26.40 ± 7.42* | 6254.1 ± 220.1* | 52.75 ± 16.25** |
| NPI1999 | 75 mg/kg | 30.98 ± 7.41 | 6393.3 ± 308.7 | 53.90 ± 20.60 |
| | intraduodenum | | | |
| Argatroban | 0.125 mg/kg i.v. | 23.55 ± 7.95* | 6259.5 ± 308.6* | 55.92 ± 11.46* |

***P < 0.001, **P < 0.01, *P < 0.05 VS. model

TABLE XIV
Effect of NPI1999 on S point in electrocardiogram of coronary artery ligation in rats

| | Dose and route | 0 min | 10 min | 30 min | |
|---|---|---|---|---|---|
| | of | after | after | after | 6 h after |
| Group | administration | ligation | ligation | ligation | ligation |
| Model | — | 0.32 ± 0.24 | 0.38 ± 0.21 | 0.34 ± 0.20 | 0.30 ± 0.21 |
| Sham-operation | — | 0.16 ± 0.15 | 0.16 ± 0.13* | 0.14 ± 0.08* | 0.18 ± 0.10 |
| NPI1999 | 0.25 mg/kg i.v. | 0.33 ± 0.17 | 0.42 ± 0.20 | 0.34 ± 0.22 | 0.24 ± 0.18 |
| NPI1999 | 0.125 mg/kg i.v. | 0.33 ± 0.12 | 0.34 ± 0.19 | 0.27 ± 0.21 | 0.08 ± 0.14* |
| NPI1999 | 75 mg/kg intraduodenum | 0.26 ± 0.13 | 0.28 ± 0.07 | 0.28 ± 0.10 | 0.18 ± 0.22 |
| Argatroban | 0.125 mg/kg i.v. | 0.35 ± 0.16 | 0.36 ± 0.20 | 0.26 ± 0.15 | 0.19 ± 0.10 |

**p < 0.01,
*p < 0.05 vs. model

TABLE XV
Effect of NPI1999 on T wave in electrocardiogram of coronary artery ligation in rats

| | | 0 min | 10 min | 30 min | |
|---|---|---|---|---|---|
| | Dose and route of | after | after | after | 6 h after |
| Group | administration | ligation | ligation | ligation | ligation |
| Model | — | 0.14 ± 0.17 | 0.15 ± 0.12 | 0.19 ± 0.11 | 0.11 ± 0.16 |
| Sham-operation | — | 0.07 ± 0.09 | 0.12 ± 0.11 | 0.15 ± 0.08 | 0.12 ± 0.10 |
| NPI1999 | 0.25 mg/kg i.v. | 0.19 ± 0.11 | 0.27 ± 0.13 | 0.26 ± 0.17 | 0.15 ± 0.11 |
| NPI1999 | 0.125 mg/kg i.v. | 0.19 ± 0.15 | 0.17 ± 0.18 | 0.18 ± 0.23 | 0.02 ± 0.16 |
| NPI1999 | 75 mg/kg intraduodenum | 0.12 ± 0.11 | 0.15 ± 0.09 | 0.18 ± 0.11 | 0.13 ± 0.11 |
| Argatroban | 0.125 mg/kg i.v. | 0.23 ± 0.16 | 0.21 ± 0.18 | 0.16 ± 0.18 | 0.11 ± 0.10 |

**p < 0.01,
*p < 0.05 vs. model

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of the following structure I: 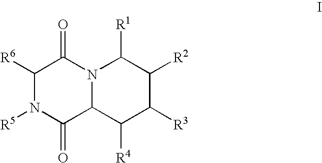
or a pharmaceutically acceptable salt and stereoisomer thereof, wherein wherein R¹, R² and R⁴ consist of a hydrogen, alkyl or aryl moiety, R³ consist of an alkyl or aryl moiety, R⁵ consists of a hydrogen, alkyl, aryl, hydroaryl, heteroaryl, hydroheteroaryl, sulfonylalkyl, sulfonylaryl, sulfonylhydroaryl, sulfonylheteroaryl or sulfonylhydroheteroaryl moiety, and R⁶ consists of a hydrogen, alkyl, aryl, hydroaryl, heteroaryl or hydroheteroaryl moiety.

2. A compound according to claim 1, wherein wherein R¹, R² and R⁴ consist of a hydrogen, alkyl or aryl moiety, R³ consist of an alkyl or aryl moiety, and wherein R⁵ consists of an alkyl, aryl, hydroaryl, heteroaryl or hydroheteroaryl moiety.

3. A compound according to claim 2, wherein R³ consists of a methyl moiety, R⁵ consists of 1,2,3,4-tetrahydro-3-methyl-8-quinolinesulfonyl, and R⁶ consists of 3-guanidinopropyl.

4. A pharmaceutical composition comprising a compound according to claim 1 as an active ingredient in association with a pharmaceutically acceptable carrier.

5. A pharmaceutical composition comprising a compound according to claim 1 in association with a pharmaceutically acceptable carrier, said pharmaceutical composition being suitable for oral administration.

6. A method for substantially reducing thrombin activity in a mammal or a human or a tissue thereof, comprising administering an effective amount of a compound according to claim 1 to said mammal, human or tissue.

7. A method for treating a coagulation disorder in a mammal or a human or a tissue thereof, comprising administering an effective amount of a compound according to claim 1 to said mammal, human or tissue.

8. A method according to claim 7, wherein the disorder consists of thrombosis or heparin-induced thrombocytopenia (HIT).

9. A method for substantially reducing thrombin activity in a mammal or a human or a tissue thereof, comprising administering an effective amount of a pharmaceutical composition according to claim 5 to said mammal, human or tissue.

10. A method for substantially enhancing fibrinolytic enzyme activity in a mammal or a human or a tissue thereof, comprising administering an effective amount of a pharmaceutical composition according to claim 5 to said mammal, human or tissue.

11. A method according to claim 10, wherein said fibrinolytic enzyme is selected from the group of urokinase, plasmin and tissue plasminogen activator (tPA).

12. A method for treating a coagulation disorder in a mammal or a human or a tissue thereof, comprising administering an effective amount of a pharmaceutical composition according to claim 5 to said mammal, human or tissue.

13. A method according to claim 12, wherein the disorder consists of thrombosis or heparin-induced thrombocytopenia (HIT).
